(19) United States
(12) Patent Application Publication
(10) Pub. No.: US 2023/0015661 A1
(43) Pub. Date: Jan. 19, 2023

(54) METHODS AND USE FOR BIOENGINEERING ENUCLEATED CELLS

(71) Applicant: The Regents of the University of California, La Jolla, CA (US)

(72) Inventors: Richard Klemke, La Jolla, CA (US); Huawei Wang, La Jolla, CA (US); Willie Pi, La Jolla, CA (US)

(21) Appl. No.: 17/944,604

(22) Filed: Sep. 14, 2022

Related U.S. Application Data
(63) Continuation of application No. 17/880,141, filed on Aug. 3, 2022.
Application No. 17/880,141 is a continuation of application No. PCT/US2021/016919, filed on Feb. 5, 2021.
(60) Provisional application No. 62/994,598, filed on Mar. 25, 2020.
(60) Provisional application No. 62/993,967, filed on Mar. 24, 2020.
(60) Provisional application No. 62/971,526, filed on Feb. 7, 2020.

(51) Int. Cl. A61K 35/13; A61P 35/00; A61K 38/20; A61K 39/395; A61K 45/06

(52) U.S. Cl. CPC A61K 35/13; A61P 35/00; A61K 38/208; A61K 39/3955; A61K 45/06; A61K 2039/54

(57) ABSTRACT

Provided are methods for treating a disease using bioengineered enucleated cells. Also provided herein are compositions comprising enucleated cells, wherein the enucleated cells have been loaded with clinically relevant biomolecules.

### CROSS-REFERENCE TO RELATED APPLICATIONS

This application is a continuation of U.S. application Ser. No. 17/880,141, filed Aug. 3, 2022, which is a continuation of International Application PCT/US2021/016919, with an international filing date of Feb. 5, 2021, which claims priority to U.S. Provisional Application Ser. No. 62/971,526, filed on Feb. 7, 2020; U.S. Provisional Application Ser. No. 62/993,967, filed on Mar. 24, 2020; and U.S. Application Ser. No. 62/994,598, filed on Mar. 25, 2020, each of which is incorporated herein by reference in its entirety.

### FEDERALLY SPONSORED RESEARCH OR DEVELOPMENT

This invention was made with Government support under Grant Nos. CA182495, and CA097022 awarded by the National Institutes of Health. The Government has certain rights in the invention.

### BACKGROUND

Current techniques and tools for cell-based therapies are often prone to unwanted and dangerous side effects, such as uncontrolled proliferation, limited engineering capability, and anti-DNA immune responses. While cell-based therapies have great potential to address critical needs in the treatment of human diseases, clinical success often faces obstacles, such as cell heterogeneity, limited engineering capability, inconsistent efficacy, poor quality control or reproducibility in large-scale manufacturing, and patient safety concerns.

### SUMMARY

The present disclosure is based, at least in part, on the generation of bioengineered enucleated cells to improve therapeutic functions and produce cell-like entities that are controllable and safe.

As elaborated on below and exemplified in the working examples, the methods for bioengineering enucleated cells designed for therapeutic use and the use of the cells offer several benefits over previous cell-based therapeutics, including, e.g., safety, defined lifespan, no risk of nuclear-encoded gene transfer to host, and effective delivery of therapeutic cargo. Other advantages of the presently claimed disclosure are described herein.

Provided herein are methods of treating a disease in a subject, the method comprising: administering to the subject a therapeutically effective amount of a composition comprising an enucleated cell genetically engineered to express at least one of an exogenous DNA molecule, an exogenous RNA molecule, an exogenous protein, or an exogenous peptide. In some embodiments, the composition further comprises a therapeutic agent. In some embodiments, the therapeutic agent comprises at least one of a small RNA, a small molecule drug, a peptide, a virus, or combinations thereof. In some embodiments, the therapeutic agent comprises a chemotherapeutic agent.

Provided herein are methods of governing immune activation in a subject, the method comprising: administering to the subject an enucleated cell, wherein the enucleated cell is genetically engineered to activate the immune system. In some embodiments, the enucleated cell is genetically engineered to express at least one exogenous protein. In some embodiments, the exogenous protein is a cell surface protein. In some embodiments, the exogenous protein is an immune activating protein. In some embodiments, the exogenous protein comprises a cytokine, IL-12, calreticulin, phosphatidylysine, phagocytosis prey-binding domain, annexin 1, OX40/OC40L, 4-1BB, B7 family members, or combinations thereof.

Provided herein are methods of governing immune recognition in a subject, the method comprising: administering to the subject an enucleated cell, wherein the enucleated cell is genetically engineered to evade recognition by the immune system. In some embodiments, the enucleated cell is genetically engineered to deplete the enucleated cell of immune recognition molecules. In some embodiments, the immune recognition molecules comprise HLA antigens, proteoglycans, sugar moieties, embryonic antigens, or combinations thereof. In some embodiments, the enucleated cell is genetically engineered to express at least one exogenous protein. In some embodiments, the exogenous protein is a cell surface protein. In some embodiments, the exogenous protein is an immune evasion molecule. In some embodiments, the exogenous protein comprises a cytokine, IL-1, IL-4, IL-6, IL-8, IL-10, TGF-β, IGF-2, VEGF, TNF-alpha, CD47, HLA-E, HLA-G, HLA-E/G, PD-1, PD-L1, TIGIT, CD112R, CTLA-4, a chemokine, chemokine ligand 1, C—C motif chemokine receptor 7, an NK inhibitor receptor, HLA-class I-specific inhibitory receptor, killer cell immunoglobulin-like receptor (KIR), NKG2A, lymphocyte activation gene-3 (LAG-3), or combinations thereof.

Provided herein are methods of identifying the presence of a disease condition in a subject, the method comprising: administering to the subject an enucleated cell genetically engineered to express at least one of an exogenous DNA molecule, an exogenous RNA molecule, an exogenous protein, or an exogenous peptide, wherein the genetically engineered enucleated cell identifies the presence or location of a disease condition. In some embodiments, the exogenous protein is an inflammation homing receptor. In some embodiments, the inflammation homing receptor directs the enucleated cell to damaged and/or inflamed tissue.

In some embodiments, the enucleated cell is derived from a natural killer (NK) cell, a macrophage, a neutrophil, a fibroblast, and adult stem cell, a mesenchymal stromal cell (MSC), an inducible pluripotent stem cell, or combinations thereof. In some embodiments, the enucleated cell is derived from a mesenchymal stromal cell (MSC).

In some embodiments, the exogenous DNA molecule comprises single-stranded DNA, double-stranded DNA, an oligonucleotide, a plasmid, a bacterial DNA molecule, a DNA virus, or combinations thereof. In some embodiments, the exogenous RNA molecule comprises messenger RNA (mRNA), small interfering RNA (siRNA), microRNA (miRNA), short hairpin RNA (shRNA), a RNA virus, or combinations thereof. In some embodiments, the exogenous protein comprises a cytokine, a growth factor, a hormone, an antibody, an enzyme, or combinations thereof.

In some embodiments, the administering comprises intravenous administration, subcutaneous administration, intraperitoneal administration, rectal administration, oral administration, or combinations thereof. In some embodiments, the administering comprises intratumoral administration.

In some embodiments, the disease comprises inflammation, an infection, a cancer, a neurological disease, an autoimmune disease, a cardiovascular disease, an ophthalmologic disease, a skeletal disease, a metabolic disease, or combinations thereof. In some embodiments, the cancer comprises multiple myeloma, glioblastoma, lymphoma, leukemia, mesothelioma, sarcoma, breast cancer, prostate cancer, ovarian cancer, pancreatic cancer, colon cancer, or combinations thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1A is a schematic of workflow for therapeutic uses of bioengineered enucleated cells (e.g., cargocytes).

FIGS. 1B and 1C are graphs showing the percentage of viable hT-MSCs/engineered enucleated cells (e.g., cargocytes) versus initial population over time. FIG. 1B, freshly isolated hT-MSCs/Cargocytes; FIG. 1C, hT-MSCs/Cargocytes thawed after 1 month cryopreservation.

FIG. 1D is a graph showing MSCs/engineered enucleated cells (e.g., cargocytes) migrated in Boyden chambers towards the indicated concentrations of SDF-1a for 2 hours. Bar graph represents the ratio of migrated cells versus loading control. Mean±SEM; n=10 independent fields from 3 biological replicates.

FIG. 1E is a bar chart showing the mean fluorescent intensity (MFI) of GFP (left) or GFP positive ratio (right) of cells and analyzed by flow cytometry.

FIG. 1F is a bar graph showing Gaussia luciferase (Gluc) activity of conditioned medium from cells 48 h post-transfection with Gluc mRNA. RLU=Relative luminescence units.

FIG. 1G is a bar graph showing the average diameter of hT-MSCs or engineered enucleated cells (e.g., cargocytes) in suspension.

FIG. 2A is a schematic of the workflow for engineered enucleated cells (e.g., cargocytes) to express IL-12 cytokine and treat triple-negative breast cancer (TNBC) in immunocompetent mouse.

FIG. 2B is a bar graph showing the level of IL-12 cytokine in established E0771 tumors detected by ELISA at indicated timepoints post-intratumoral injection. PBS, vehicle control; MSC-IL-12/Cargocyte-IL-12, hT-MSC or Cargocyte transfected with IL-12 mRNA.

FIG. 2C is a graph showing the fold change (Log2) of the indicated mRNA markers compared to PBS group in mice treated as in FIG. 2B. Tumors were harvested and analyzed by real-time RT-PCR at 48 hr post-i.t. injection.

FIG. 2D are bar graphs showing the harvested tumors analyzed by flow cytometry. Mice were treated and tumors were harvested and analyzed by flow cytometry. % CD8⁺ T cells, 100×CD8⁺CD4⁻CD3⁺CD45⁺/CD45⁺; % CD4⁺ T cells, 100×CD8⁻CD4⁺CD3⁺CD45⁺/CD45⁺; M1Φ/M2Φ, CD45⁺Ly6c⁻F4/80⁺MHCII^{high}/CD45⁺Ly6c⁻F4/80⁺MHC II^{low}; % Foxp3⁺ Treg cells, 100×CD45.2⁺CD4⁺CD25⁺Foxp3⁺/CD45⁺; % NK cells, 100×CD45⁺CD3⁻ NK1.1⁺/CD45⁺; % CD45⁺ cells, 100×CD45⁺/total cells.

FIG. 2E is a graph showing the timeline for intratumoral injection of MSC-IL-12/Cargocyte-IL-12 to pre-established E0771 tumors and intraperitoneal injection of anti-PD-1 antibody (upper) and the Kaplan-Meier survival curve for these mice (lower).

FIG. 2F is a graph showing tumor growth curve for mice that survived in FIG. 2E and re-challenged with E0771 cells.

FIG. 2G is a graph showing the fold change (Log2) of the indicated mRNA markers (IDO1, Indoleamine 2, 3-dioxygenase 1; PD-L1, Programmed death-ligand 1) compared to mock-treated control. Control hT-MSC, irradiated MSC (30 Gy), and FACS-sorted engineered enucleated cells (e.g., cargocytes) were stimulated by human interferon gamma (IFN-γ) for 6 hours, and analyzed by real-time RT-PCR.

FIG. 3A is a schematic of the workflow for using engineered enucleated cells (e.g., cargocytes) to treat LPS-induced acute ear inflammation in a mouse model.

FIG. 3B is a bar graph showing the number of DiD⁺RFP⁺ double-positive cells out of 1E5 total cells harvested from mouse lung 24 hr post-intravenous injection and detected by flow cytometry.

FIG. 3C is a bar graph showing the number of DiD⁺F4/80⁻ cells out of 1E5 total cells harvested from mouse ears 24 h post-injection and detected by flow cytometry. Mice were injected with LPS in the right ear and saline in the left, followed by i.v. injection of DiD-labeled MSCs or Cargocytes 6 hr later. D1 MSC/Cargocyte, mouse D1 MSC/Cargocyte; 3D-MSC/Cargocyte, 3D-cultured parental MSC/Cargocyte; 3D-MSC^{Tri-E C19}, Triple (CXCR4/CCR2/PSGL-1) engineered MSC Clone 19; 3D-Cargocyte^{Tri-E C19} 3D-MSC^{Tri-E C19-} derived Cargocyte.

FIG. 3D is a bar graph showing the level of human IL-10 protein detected by ELISA from indicated mouse ears at 24 hr post-injection, where mice treated as in FIG. 3C were i.v.-injected with indicated cells or Cargocytes after human IL-10 mRNA transfection.

FIG. 3E is light microscopy images of ears from mice treated as in FIG. 3C and harvested at 48 hr post-injection and processed for hematoxylin and eosin staining.

FIG. 3F is a graph showing change in ear thickness as measured by digital micrometer prior to LPS/Saline injections and 48 hr after cell/engineered enucleated cell (e.g., cargocyte) injection.

FIG. 3G is a graph showing the fold change (Log2) of the indicated mRNA markers between LPS-treated (right) and saline-treated (left) ears, where mice treated as in FIG. 3F had ears harvested and analyzed by real-time RT-PCR 48 hr after LPS injection.

FIG. 4 is fluorescent images of MSCs or engineered enucleated cells (e.g., cargocyte) stained with indicated subcellular organelle antibodies (arrows) and DAPI. Mitochondria, anti-AIF (Apoptosis-inducing factor); Lysosome, anti-LAMP1 (Lysosome-associated membrane protein 1); Golgi, anti-RCAS1 (Receptor binding cancer antigen expressed on SiSo cells); Endoplasmic Reticulum (ER), anti-PDI (Protein disulfide isomerase); Endosome, anti-EEA1 (Early Endosome Antigen 1). Arrows point to indicated organelles. Scale bar=50 μm.

FIG. 5A is a bar graph representing the ratio of migrated MSCs/engineered enucleated cells (e.g., cargocytes) versus loading control (MSCs/engineered enucleated cells (e.g., cargocytes) seeded on fibronectin-coated plates).

FIG. 5B is a bar graph representing the ratio of migrated MSCs/engineered enucleated cells (e.g., cargocytes) versus loading control, where MSCs/Cargocytes migrated in Boyden chambers towards PDGF-AB gradients.

FIG. 5C is a bar graph representing the ratio of migrated MSCs/engineered enucleated cells (e.g., cargocytes) versus loading control.

FIG. 5D is a bar graph represents number of attached cells per field, where MSCs and Cargocytes were allowed to attach to fibronectin-coated 24 well plate in serum free media with 0.25% BSA (2E4 cells per well) for 2 hours. The attached cells were stained with crystal violet and were counted using light microscopy at 400× magnification.

FIG. 6A is a bar graph showing the recovery rate (percentage viable cells out of the input population) for MSCs/engineered enucleated cells (e.g., cargocytes) thawed after 1 month of cryopreservation.

FIG. 6B is a bar graph representing the ratio of migrated MSCs or engineered enucleated cells (e.g., cargocytes) versus loading control, where recovered MSCs or Cargocytes migrated in Boyden chambers towards FBS gradients.

FIG. 7A is a schematic design of the mouse IL-12a and IL-12b mRNAs synthesized in vitro. Kozak sequence was added in front of the start codon of the IL-12 mRNA coding region (CDS). 5′UTR and 3′UTR of mouse alpha globin mRNA were added respectively to the 5′ and 3′ end of CDS. An artificial 5′Cap was added to the 5′ end of mRNAs and the pseudouridine modification was engineered to increase mRNA stability.

FIG. 7B is a graph showing the secreted IL-12 concentration in conditioned media of IL-12 transfected MSCs (MSC-IL-12), Cargocytes (Cargocyte-IL-12) or non-transfected cells (Control MSC).

FIG. 7C shows western blot images where mouse splenocytes were treated with indicated conditioned media or recombinant mouse IL-12 (p70) protein (10 ng/ml) for 30 mins. The phosphorylation of Stat4 was determined by western blot.

FIG. 7D is a bar graph showing the concentration of secreted IL-12 cytokine in the mouse plasma as determined by ELISA, where mice were treated as in FIG. 2B.

FIG. 8A are bar graphs showing the average diameter of indicated MSCs or engineered enucleated cells (e.g., cargocytes) in suspension.

FIG. 8B is a bar graph showing the average time required for cells to migrate through an individual microfluidic constriction. Data for both confined (<2 μm×5 μm) and unconfined (151 μm×5 μm) constrictions are shown.

FIG. 8C are bar graphs showing the fold change (Log2) of the indicated mRNA markers in LPS-treated ears at indicated time points and normalized to saline-treated ear (control), where mouse ears were harvested and analyzed by real-time RT-PCR at 6 hrs or 24 hrs after LPS injection.

FIGS. 9A and 9B are bar graphs representing the ratio of migrated MSCs/Cargocytes versus loading control (MSCs or Cargocytes seeded onto fibronectin-coated plates), where MSCs/Cargocytes migrated in Boyden chambers towards the indicated chemokine gradient for 2 hours.

FIG. 9C is a bar graph showing the average numbers of DiD+F4/80− MSCs or Cargocytes out of 1E5 total cell harvested from mouse ears at 24 h post injection and detected by flow cytometry, where mice were treated and i.v. injected with indicated cells.

FIG. 9D is a bar graph showing the number of DiD+F4/80− cells out of 1E5 total cell harvested from mouse lung at 24 hr post-injection and detected by flow cytometry. D1 MSC/Cargocyte, mouse D1 MSC/Cargocyte; 3D-MSC/Cargocyte, 3D-cultured parental MSC/Cargocyte; 3D-MSC^{Tri-E C19}, Triple (CXCR4/CCR2/PSGL-1) engineered MSC Clone 19; 3D-Cargocyte^{Tri-E C19} 3D-MSC^{Tri-E C19} derived Cargocyte.

FIG. 10 are graphs showing quantitative analysis of bioluminescence imaging signal intensity (photons/sec/cm2/steradian) from indicated mouse organs at different time points using the software LivingImage V4.1.

FIG. 11A are graphs showing quantitative analysis of bioluminescence imaging signal intensity (photons/sec/cm2/steradian) from peeled mouse ears at different time points using the software LivingImage V4.1.

FIG. 11B is a bar graph showing firefly luciferase (Fluc) activity measured by SpectraMax M2e of conditioned medium from cells at indicated time points post-transfection with Fluc mRNA.

FIG. 12A is a schematic design of the human IL-10 mRNA synthesized in vitro. Kozak sequence was added in front of the start codon of the IL-10 mRNA coding region (CDS). 5′UTR and 3′UTR of mouse alpha globin mRNA were added respectively to the 5′ and 3′ end of CDS. An artificial 5′Cap was added to the 5′ end of mRNAs and the pseudouridine modification was engineered to increase mRNA stability.

FIG. 12B is a graph showing the secreted IL-10 concentration in conditioned media of IL-10 transfected MSCs (MSC-IL-10), Cargocytes (Cargocyte-IL-10), non-transfected cells (MSC only) or control media.

FIG. 12C is a western blot image showing mouse RAW macrophage cells were treated with indicated conditioned media or recombinant IL-10 protein (1 ng/ml) for 30 mins and the phosphorylation of Stat3 was determined by western blot.

FIG. 12D is a bar graph showing the secreted IL-10 concentration in conditioned media of D1 MSC or D1 Cargocytes 24 hr after IL-10 mRNA transfection.

FIG. 12E is a bar graph showing the concentration of secreted IL-10 cytokine in the mouse plasma as determined by ELISA.

FIG. 12F is a graph showing the secreted IL-10 concentration measured by ELISA in conditioned media of IL-10 mRNA transfected MSCs (MSC-IL-10) and Cargocytes (Cargocyte-IL-10), non-transfected cells (hTMSC only) or control media.

FIG. 13A is a graph showing the percentage of viable MSCs or Cargocytes versus initial population over time. Sorted MSC Control, FACS sorted MSC^{H2B-GFP}; Cargocytes and Karyoplast/MSC were separated from enucleated MSC^{H2B-GFP} based on GFP expression.

FIG. 13B is a bar graph representing the ratio of migrated MSCs or Cargocytes versus loading control where MSCs^{H2B-GFP} or sorted Cargocytes^{H2B-GFP} migrated in Boyden chambers towards FBS gradients.

FIG. 13C is a bar graph representing the cell migration index (migrated Cargocytes versus loading control) where MSCs^{H2B-GFP} or sorted Cargocytes^{H2B-GFP} migrated in Boyden chambers towards FBS gradients.

FIG. 13D is a graph showing the percentage of viable hT-MSCs/Cargocytes versus initial population over time where hT-MSCs/Cargocytes thawed after 1 month cryopreservation.

FIG. 13E is a bar graph showing the recovery rate (percentage viable cells out of the input population) for MSCs/Cargocytes thawed after 1 month of cryopreservation.

FIG. 13F is a bar graph representing the cell migration index (migrated MSCs/Cargocytes versus loading control) where recovered MSCs or Cargocytes migrated in Boyden chambers towards FBS gradients.

FIG. 14 is a bar graph representing the ratio of induced migration (with chemokine gradient) versus background migration (without chemokine gradient) where MSCs/cargocytes migrated in Boyden chambers towards the indicated chemokine gradient for 2 hours.

FIG. 15A is a schematic of E0771 TNBC survival experiments: Cargocytes transfected with IL-12 mRNA (CA-IL-12) were intratumorally (IT) injected every 2-3 days into mice bearing subcutaneous (SQ) E0771 tumors. Control mice received IT PBS. Twenty-four hours after the third dose, either anti-PD-1 or control anti-IgG isotype was administered intraperitoneally (IP). The next week, a final Cargocyte IL-12 or PBS dose was administered IT followed by anti-PD-1 or anti-IgG IP the next day. Tumors were measured and animals euthanized when tumors grew >2 cm diameter.

FIG. 15B is a graph of fold change in animal weight during the treatment phase of survival experiments. Arrows=administration of IT CA-IL-12 or PBS; arrowheads=IP anti-PD-1 or anti-IgG administration.

FIG. 15C is a table showing analysis of the indicated inflammation cytokines by ELISA where MSCs or Cargocytes were intravenously (IV) injected into mice. Serum was collected 2 and 24 hours post-IV injection.

FIG. 15D is a graph showing fold change in tumor size for each side (injected and contralateral/uninjected) where in a separate experiment, animals were bilaterally injected with E0771 cells and then IT injected unilaterally with 3 doses of CA-IL-12 or PBS.

FIG. 16A is a bar graph representing the MFI change of LDV-FITC binding intensity before and after SDF-1α treatment. MFI ratio=(MFI^{LDV-FITC+SDF-1α}-MFI^{unstained control})/(MFI^{LDVFITC}-MFI^{unstained control}).

FIG. 16B are bar graphs representing the adherent cell numbers per field (100× magnification). TNF-α, HUVECs pre-treated with 10 ng/ml TNF-α for 6 hours. SDF-1a, 500 ng/ml SDF-1α; a-PSGL-1, 10 μg/ml anti-PSGL-1 antibody pre-treatment; a-VLA-4, 10 μg/ml anti-VLA-4 antibody pre-treatment.

FIG. 16C are bar graphs representing the fold change of induced migration (with chemokine gradient) versus background migration (without chemokine gradient) where MSCs/Cargocytes migrated in Boyden chambers towards indicated chemokine gradients for 2 hours.

FIG. 17A is a bar graph showing the number of DiD+F4/80− cells out of 1E5 total cells harvested from mouse pancreas 16 hr post-injection and detected by flow cytometry. Mice with Caerulein-induced AP were i.v.-injected with indicated treatments where mouse tissues were harvested 16 hr post-injection. Acute pancreatitis was induced by intraperitoneal (i.p.) injection of Caerulein in BalB/c mice, followed by i.v. injection of DiD-labeled MSCs or Cargocytes.

FIG. 17B is a bar graph showing the level of human IL-10 protein detected by ELISA from mouse pancreas from indicated treatment.

FIG. 17C is a bar graph showing the relative mRNA expression of Ccl2 (upper) and TNF-α (lower) detected by real-time RT-PCR in the mouse pancreas from indicated treatment. Graphs show the fold change (Log2) of the indicated mRNA markers normalized to no Caerulein treatment group.

FIG. 17D is a bar graph showing the lipase activity (upper) and amylase activity (lower) detected in the mouse serum from indicated treatment.

FIG. 17E is a bar graph showing histological analysis of pancreas. The severity of edema (upper) and necrosis (lower) were graded from 0 to 3 using established criteria.

FIG. 18A are graphs showing the fold change (Log2) of the indicated mRNA expression in the pancreas.

FIG. 18B are bar graphs showing the number of DiD+F4/80- cells out of 1E5 total cells harvested from mouse lung or liver 16 hr post-injection and detected by flow cytometry. FIG. 18C is a bar graph showing the secreted IL-10 concentration in conditioned media of BM-MSC or BM-MSC transfected IL-10 mRNA transfection at 24 hr post transfection. FIG. 18D is a bar graph showing the secreted IL-10 concentration at 24 hr post treatment in conditioned media of HEK293 cells treated with exosome only or exosome-loaded with IL-10 mRNA.

FIG. 18E are bar graphs showing the level of human IL-10 protein detected by ELISA from mouse plasma or tissue from indicated treatment.

FIG. 18F are bar graphs showing the relative mRNA expression of IL-6 (upper) and IL-1β (lower) detected by real-time RT-PCR in the mouse pancreas from indicated treatment. Graphs show the fold change (Log2) of the indicated mRNA markers normalized to no Caerulein treatment group.

FIG. 18G is a bar graph showing histological analysis of pancreas. The severity of inflammatory cell infiltration were graded from 0 to 3 using established criteria.

### DETAILED DESCRIPTION

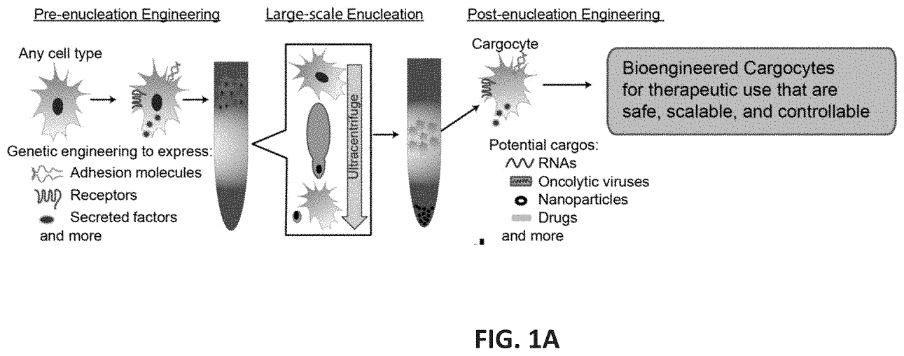

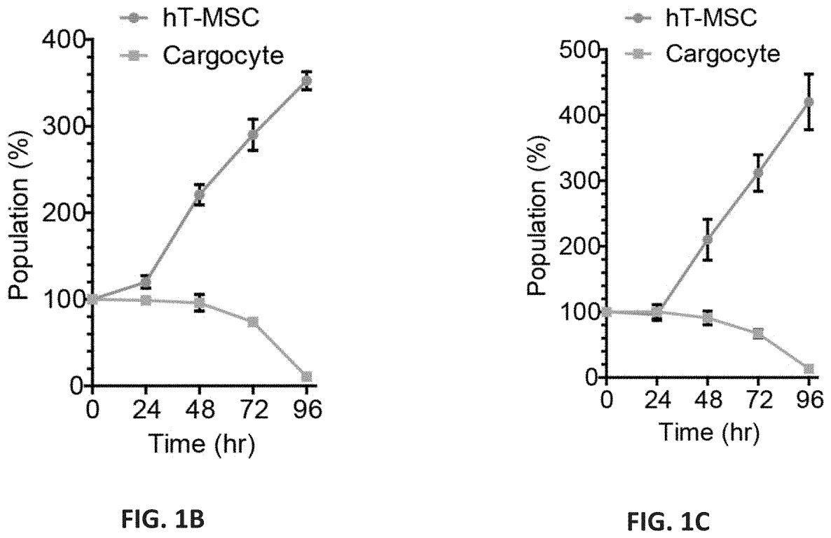

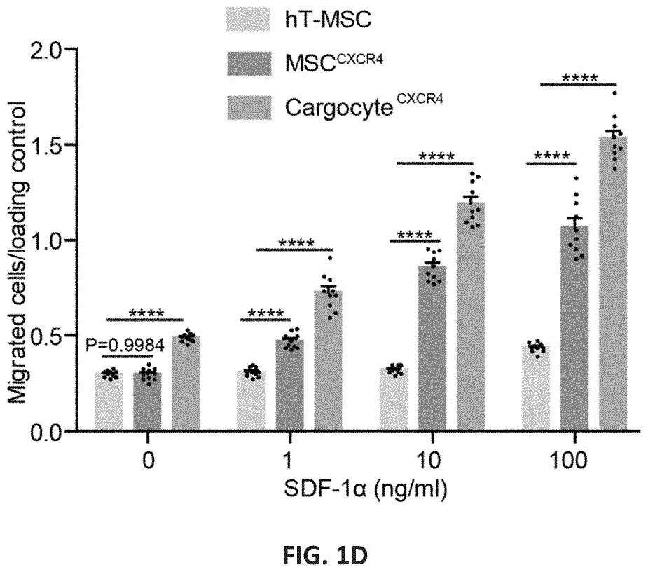

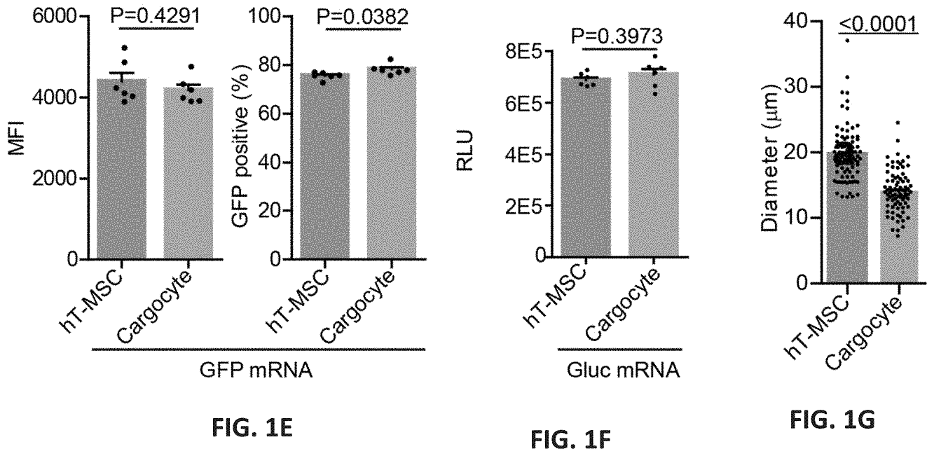

This disclosure describes methods and uses for cell-based therapies with genetically engineered enucleated cells. In practice, cells can be genetically engineered to improve therapeutic functions and are enucleated to produce cell-like entities that are controllable and safe (FIG. 1A). Further, manufacturing significant numbers of therapeutic cells for clinical applications is limiting to many cell-based therapies, especially in the stem cell field. Therefore, there is significant commercial interest to use immortalized cells (e.g., hT-MSC, viruses and oncogenes) to increase manufacturing capabilities, because it is robust and cost-effective. However, immortalized cells can cause cancer, and thus can be too dangerous for therapeutic applications. The present disclosure allows for the use of immortalized cells or even cancerous cells for therapeutic applications, because they are rendered safe by enucleation prior to administration.

### Enucleated Cells

Bioengineered enucleated cells can be designed for therapeutic use by performing important cellular functions after enucleation, having a defined lifespan, exhibiting therapeutic functions, and being amenable to multi-layered engineering and large-scale manufacturing. As used herein, “enucleation” is the rendering of a cell to a non-replicative state, either through inactivation or removal of the nucleus. In some embodiments, cells can be treated with cytochalasin to soften the cortical actin cytoskeleton. The nucleus is then physically extracted from the cell body by high-speed centrifugation in gradients of Ficoll to generate an enucleated cell. Because enucleated cells and intact nucleated cells sediment to different layers in the Ficoll gradient, enucleated cells can be easily isolated and prepared for therapeutic purposes or fusion to other cells (e.g., nucleated or enucleated cells). In some embodiments, the enucleation process is clinically scalable to process tens of millions of cells.

In some embodiments, enucleated cells can be used as a disease-homing vehicle to deliver clinically relevant cargos/payloads to treat various diseases (e.g., any of the diseases described herein). In some embodiments, enucleated cells loaded with cargos, payloads, or biomolecules can be referred to as “cargocytes”. In some embodiments, cargocytes can refer to bioengineered enucleated cells designed for therapeutic use. In some embodiments, enucleated cells possess significant therapeutic value because they remain viable, do not differentiate into other cell types, secrete bioactive proteins, can physically migrate/home for 3-4 days, can be extensively engineered ex vivo to perform specific therapeutic functions, and can be fused to the same or other cell types to transfer desirable production, natural or engineered. Therefore, enucleated cells have wide utility as a cellular vehicle to deliver therapeutic biomolecules and disease-targeting cargos including, but not limited to, chemotherapeutic drugs (e.g., doxorubicin), genes, viruses, bacteria, mRNAs, shRNAs, siRNA, peptides, plasmids, and nanoparticles. In some embodiments, enucleated cells enable the generation of a safe (e.g., no unwanted DNA is transferred to the subject), and controllable (e.g., cell death occurs in precisely 3-4 days) cell-based carrier that can be genetically engineered to deliver specific disease-fighting and health promoting cargos to humans or animals.

In some embodiments, an enucleated cell (e.g., cargocyte) is genetically engineered and designed for therapeutic use. As used herein, “genetically engineered,” in reference to cells, refers to a cell that comprises a nucleic acid sequence (e.g., DNA, RNA, or mRNA) that is not present in, or is present at a different level than, an otherwise similar cell under similar conditions that is not engineered (e.g., compared to RBCs, which are derived from erythroblasts, an enucleated cell (e.g., cargocyte) can be derived from any type of nucleated cell, including, but not limited to iPSC (induced pluripotent stem cells), any immortalized cell, stem cells, primary cells an exogenous DNA molecule, or an exogenous RNA molecule), or a cell that comprises a polypeptide expressed from said nucleic acid (e.g., an exogenous protein, or an exogenous polypeptide). In some embodiments, a genetically engineered cell has been altered from its native state by the introduction of an exogenous nucleic acid, or is the progeny of such an altered cell. In some embodiments, a genetically engineered cell comprises an exogenous nucleic acid (e.g., DNA, RNA, or mRNA). In some embodiments, the enucleated cell is engineered to express at least one (e.g., one or more, two or more, three or more, four or more, five or more, or six or more) of an exogenous DNA molecule, an exogenous RNA molecule, an exogenous polypeptide, or an exogenous protein, or combinations thereof. In some embodiments, the enucleated cell is engineered to simultaneously express at least two or more (e.g., three or more, four or more, five or more, or six or more) of an exogenous DNA molecule, an exogenous RNA molecule, an exogenous polypeptide, or an exogenous protein, or combinations thereof. In some embodiments, the exogenous DNA molecule is a single-stranded DNA, a double-stranded DNA, an oligonucleotide, a plasmid, a bacterial DNA molecule, a DNA virus, or combinations thereof. In some embodiments, the exogenous RNA molecule is messenger RNA (mRNA), small interfering RNA (siRNA), microRNA (miRNA), short hairpin RNA (shRNA), a RNA virus, or combinations thereof. In some embodiments, the exogenous protein is a cytokine, a growth factor, a hormone, an antibody, an enzyme, or combinations thereof.

In some embodiments, enucleated cells can be derived from a variety of different cell types. In some embodiments, enucleated cells can be derived from any nucleated cell type that maintains a nucleus throughout its lifespan or does not naturally enucleate. In some embodiments, an enucleated cell can be derived from a normal cell line. In some embodiments, an enucleated cell can be derived from a cancer cell line. In some embodiments, an enucleated cell can be derived from therapeutic cells obtained from the immune system. For example, an enucleated cell can be derived from a mesenchymal stromal cell (MSC), a natural killer (NK) cell, a macrophage, a neutrophil, a lymphocyte, a mast cell, a basophil, an eosinophil, and/or a fibroblast. In some embodiments, an enucleated cell is derived from a mesenchymal stromal cell (MSC). In some embodiments, an enucleated cell is derived from hTERT-immortalized adipose-derived MSCs (hT-MSC) wherein MSCs have proven therapeutic potential in clinical studies and the immortalized phenotype provides a homogenous cell population with consistent characteristics, which facilitates further bioengineering. In some embodiments, an enucleated cell can be derived from an adult stem cell and/or an inducible pluripotent stem cell (iPSC).

Some cell types do not have a nucleus, e.g., red blood cells. Further, exosomes and small cellular membrane vesicles derived from therapeutic cells can act as delivery vesicles, but are markedly different than the enucleated cells of this disclosure. Enucleated cells of this disclosure (e.g., cargocytes) are different from RBCs, exosomes, and small cellular membrane vesicles. These types of delivery vesicles do not have the cellular organelles needed to produce and secrete exogenous proteins (e.g., ER/Golgi, mitochondrial, endosome, lysosome, cytoskeleton, etc.). Thus, enucleated cells of the disclosure can function like nucleated cells and exhibit critical biological functions such as adhesion, tunneling nanotube formation, actin-mediated spreading (2D and 3D), migration, chemoattractant gradient sensing, mitochondrial transfer, mRNA translation, protein synthesis, and secretion of exosomes and other bioactive molecules. One or more of these functions may not be exhibited by exosomes, small cellular membrane vesicles, RBCs, or other similar delivery-only vesicles.

In some embodiments, enucleation efficiency describes the percentage of cells in a population that have been successfully enucleated through the methods described here or otherwise known in the art. In some embodiments, the enucleation efficiency of cells can be over 95% (e.g., 96%, 97%, 98%, 99%, or 100%) efficient. In some embodiments, a recovery rate refers to the percentage of viable cells out of an input population. In some embodiments, enucleated cells can be generated with an at least 80% (e.g., at least 85%, at least 90%, at least 95%, at least 96%, at least 97%, at least 98%, at least 99%, or 100%) recovery rate. In some embodiments, over 95% enucleation efficiency is achieved for hT-MSCs. In some embodiments, the methods of the disclosure yield an 80-90% recovery rate.

As used herein, the term “substantially the same”, when used herein with respect to cell structure, can refer to a cell relative to a reference cell sharing at least the functional subcellular organelles. For example, an enucleated cell may exhibit substantially the same cell structure as a parental cell if the two cells contain the same functional subcellular organelles. In some embodiments, the enucleated cell contains the same functional subcellular organelles as a parental cell wherein the functional subcellular organelles comprise at least one of the Golgi, Endoplasmic Reticulum, mitochondria, lysosomes, ribosomes, endosomes, or combinations thereof.

The term “substantially the same”, when used herein with respect to cell function, can refer to a cell relative to a reference cell exhibiting similar functional characteristics. For example, an enucleated cell may retain same surface marker protein expression. In some embodiments, the enucleated cell has similar zeta potential as a parental (e.g., nucleated) cell. In some embodiments, the enucleated cell membrane receptors and migration and invasion machineries are fully functional, exhibiting similar functionality as a parental cell. In some embodiments, the enucleated cell actively produces and secretes the same extracellular vesicles as those produced by a parental cell.

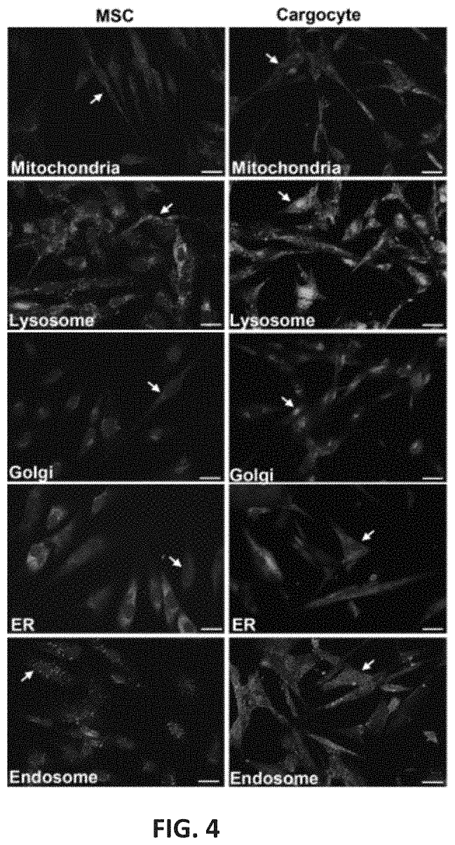

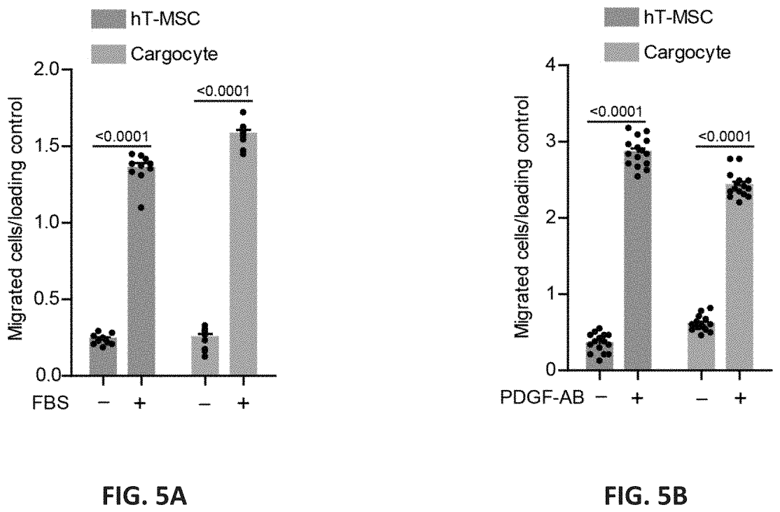

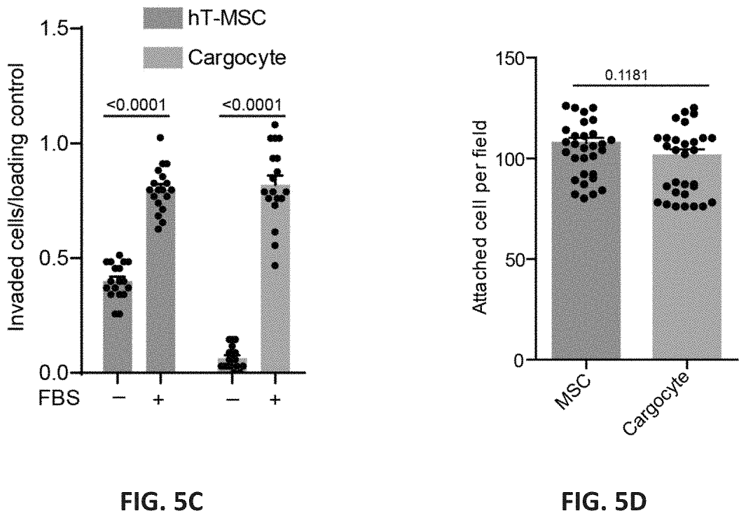

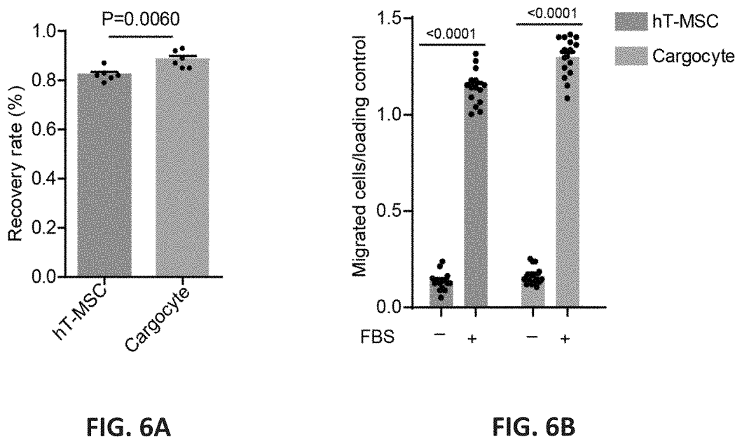

In some embodiments, enucleated cells readily attach to tissue culture plates with well-organized cytoskeletal structure. In some embodiments, enucleated cells are viable for up to 72 hours post-enucleation. In some embodiments, enucleated cells can contain crucial and functional subcellular organelles, including, but not limited to, Golgi, Endoplasmic Reticulum (ER), mitochondria, lysosomes, and endosomes (FIG. 4). In some embodiments, enucleated cells can retain surface marker protein (e.g., CXCR4, CCR2, PSGL-1, CD44, CD90, CD105, CD106, CD166, or Stro-1) expression for at least 48 hours and have similar zeta potential as parental (nucleated) cells. In some embodiments, enucleated cells sense and migrate towards chemoattractants in vitro, and invade through 3D Matrigel-coated membranes towards FBS gradients (FIGS. 5A-5D), suggesting that enucleated cell membrane receptors and migration and invasion machineries are fully functional in the enucleated cells. In some embodiments, extracellular vesicles (EVs) isolated from conditioned media (CM) of enucleated cells can have similar characteristic morphology by electron microscopy, similar size distribution, and similar amount produced as measured by BCA assay. This suggests that enucleated cells can actively produce and secrete EVs that do not differ significantly from those produced by parental cells. In some embodiments, enucleated cells show recovery from cryopreservation at higher rates than parental cells, and maintain both viability and migration ability after thawing (FIGS. 6A and 6B), which facilitates the logistics of storage and delivery in clinical applications. In some embodiments, enucleated cells retain critical cell structures and functions, and therefore have potential for therapeutic applications.

In some embodiments, cell-based therapeutics use normal or engineered nucleated cells. In some embodiments, cell-based therapies irradiate cells prior to patient injection in order to prevent cell proliferation and induced lethal DNA-damage. However, this approach induces mutations and produces significant amounts of reactive oxygen species that irreversibly damage cellular proteins and DNA, which can release large amounts of damaged/mutated DNA into the body of a subject. Such products can be dangerous if they integrate into other cells and/or induce an unwanted anti-DNA immune response. Irradiated cells are also dangerous because they can transfer their mutated DNA and genes to host cells by cell-cell fusion. Compared to cellular irradiation, removing the entire nucleus from a cell is a less damaging and significantly safer method for limiting cellular lifespan that precludes any introduction of nuclear DNA into a subject. Furthermore, many stem cells such as mesenchymal stem cells (MSCs) are highly resistant to radiation-induced death, and therefore can not be rendered safe using this method.

In some embodiments, therapeutic cells can be engineered with a drug-inducible suicide switch to limit cellular lifespan. However, activation of the switch in vivo requires injecting a subject with potent and potentially harmful drugs with unwanted side effects. While this method induces suicide in culture cells (<95%), it is expected to be inefficient when translated into the clinic. Moreover, the death of the therapeutic cell released large amounts of DNA (e.g., normal or genetically altered DNA), which can integrate into host cells or induce a dangerous systemic anti-DNA immune response. If the cell mutates and loses/inactivates the suicide switch, it becomes an uncontrollable mutant cell. In addition, these cells can fuse with host cells in the subject, and therefore transfer mutant DNA. Such fused cells are dangerous because not all host cells inherit the suicide gene, but can inherit some of the therapeutic cell's genes/DNA during chromosomal reorganization and cell hybridization. In addition, for the same reason, therapeutic cells with suicide switches can not be used as cell fusion partners in vitro.

Another method to limit therapeutic cell lifespan is heat-induced death. However, this causes severe damage that terminates crucial biological functions necessary for therapeutic use. Unlike enucleated cells, these cells can still transfer DNA to the subject since they retain their nucleus and all genetic material. Numerous chemicals inhibit cell proliferation and/or cause cell death prior to therapeutic use, including, but not limited to, chemotherapeutic drugs or mitomycin C. However, such drugs have significant off-target effects that significantly damage the cell, and are unwanted for clinical applications due to high toxicities. Many anti-proliferative and death-inducing drugs do not effectively inhibit 100% if the cells due to resistance, and unlike enucleated cells, many drug effects are revisable. Thus, this approach is not suitable to prevent cell growth of immortalized/cancer cells in vivo.

The present disclosure provides methods for producing engineered enucleated cells (e.g., cargocytes) designed for therapeutic use. In some embodiments, enucleated cells are produced with either natural or inducible expression and/or uptake of biomolecules with therapeutic functions including, but not limited to, DNA, RNA (e.g., mRNA, shRNA, siRNA, miRNA), nanoparticles, peptides, proteins, plasmids, viruses, and small molecule drugs. In some embodiments, bioengineering approaches improve enucleated cell function. In some embodiments, parental cells (e.g., nucleated cells) are genetically engineered before enucleation (e.g., pre-enucleation). In some embodiments, parental cells are genetically engineered after enucleation (e.g., post-enucleation).

In some embodiments, enucleated cells (e.g., cargocytes) are engineered to produce biomolecules (secreted, intracellular, and natural and inducible) exogenously including, but not limited to, DNA/genes, RNA (e.g., mRNA, shRNA, siRNA, miRNA), nanoparticles, peptides, proteins, and plasmids, bacteria, viruses, small molecule drugs, ions, cytokines, growth factors, and hormones. In some embodiments, enucleated cells produce therapeutic levels of a bioactive protein or an immune stimulator.

In some embodiments, parental cells are genetically engineered to produce biomolecules (secreted, intracellular, and natural and inducible) exogenously before enucleation. In some embodiments, parental cells are genetically engineered to produce biomolecules exogenously including, but not limited to, DNA/genes, RNA (e.g., mRNA, shRNA, siRNA, miRNA), nanoparticles, peptides, proteins, and plasmids, bacteria, viruses, small molecule drugs, ions, cytokines, growth factors, and hormones. In some embodiments, parental cells are genetically engineered to produce therapeutic levels of a bioactive protein or an immune stimulator. In some embodiments, parental cells are genetically engineered to produce tumor trophic proteins.

In some embodiments, enucleated cells can be used as a vehicle to deliver therapeutic biologics (e.g., therapeutic cargos) including, but not limited to, DNA/genes, RNA (e.g., mRNA, shRNA, siRNA, miRNA), nanoparticles, peptides, proteins, plasmids, viruses, and small molecule drugs. Unlike nucleated cells, enucleated cells can be loaded with high doses of DNA-damaging/gene targeting agents for delivery to patients as a therapeutic against cancer or other diseases. In some embodiments, the DNA-damaging/gene targeting agents include, but are not limited to, DNA-damaging chemotherapeutic drugs, DNA-integrating viruses, oncolytic viruses, and gene therapy applications.

In some embodiments, therapeutic enucleated cells (natural or engineered) can be used as fusion partners to other cells (therapeutic or natural) to enhance and/or transfer biomolecules (secreted, intracellular, and natural and inducible) including, but not limited to, DNA/genes, RNA (mRNA, shRNA, siRNA, miRNA), nanoparticles, peptides, proteins, and plasmids, bacteria, viruses, small molecule drugs, ions, cytokines, growth factors, and hormones. Unlike nucleated cells, the fusion of enucleated cells to the same or another cell type of similar or different origin generates a unique cell hybrid that lacks problematic nuclear transfer, while maintaining desirable therapeutic attributes including, but not limited to, cell surface proteins, signal transduction molecules, secreted proteins, and epigenetic changes.

In some embodiments, enucleated cells can be used as biosensors and signal transduction indicators of biological processes and disease states. In some embodiments, because enucleated cells cannot undergo DNA damage-induced apoptotic death, they can be used in combination with apoptotic-inducing and/or DNA toxic/targeting agents for treatment of cancer and other diseases.

Enucleated cells are smaller than their nucleated counterparts and for this reason can migrate better through small openings in the vasculature and tissue parenchyma. In addition, removing the large dense nucleus alleviates a major physical barrier allowing the cell to move freely through small openings in the vessels and tissue parenchyma. Therefore, enucleated cells have improved bio-distribution in the body and movement into target tissues. In some embodiments, an enucleated cell is at least 1 μm in diameter. In some embodiments, an enucleated cell is greater than 1 μm in diameter. In some embodiments, an enucleated cell is 1-100 μm in diameter (e.g., 1-90 μm, 1-80 μm, 1-70 μm, 1-60 μm, 1-50 μm, 1-40 μm, 1-30 μm, 1-20 μm, 1-10 μm, 1-5 μm, 5-100 μm, 5-90 μm, 5-80 μm, 5-70 μm, 5-60 μm, 5-50 μm, 5-40 μm, 5-30 μm, 5-20 μm, 5-10 μm, 10-100 μm, 10-90 μm, 10-80 μm, 10-70 μm, 10-60 μm, 10-50 μm, 10-40 μm, 10-30 μm, 10-20 μm, 20-100 μm, 20-90 μm, 20-80 μm, 20-70 μm, 20-60 μm, 20-50 μm, 20-40 μm, 20-30 μm, 30-100 μm, 30-90 μm, 30-80 μm, 30-70 μm, 30-60 μm, 30-50 μm, 30-40 μm, 40-100 μm, 40-90 μm, 40-80 μm, 40-70 μm, 40-60 μm, 40-50 μm, 50-100 μm, 50-90 μm, 50-80 μm, 50-70 μm, 50-60 μm, 60-100 μm, 60-90 μm, 60-80 μm, 60-70 μm, 70-100 μm, 70-90 μm, 70-80 μm, 80-100 μm, 80-90 μm, or 90-100 μm). In some embodiments, some enucleated cells can advantageously be small enough to allow for better biodistribution or to be less likely to be trapped in the lungs of a subject.

In some embodiments, a genetically engineered enucleated cell has a defined life span of less than 1 hour to 14 days (e.g., less than 1 hour, less than 6 hours, less than 12 hours, less than 1 day, less than 2 days, less than 3 days, less than 4 days, less than 5 days, less than 6 days, less than 7 days, less than 8 days, less than 9 days, less than 10 days, less than 11 days, less than 13 days, less than 14 days, 1 to 14 days, 1 to 12 days, 1 to 10 days, 1 to 9 days, 1 to 8 days, 1 to 7 days, 1 to 6 days, 1 to 5 days, 1 to 4 days, 1 to 3 days, 1 to 2 days, 2 to 14 days, 2 to 12 days, 2 to 10 days, 2 to 9 days, 2 to 8 days, 2 to 7 days, 2 to 6 days, 2 to 5 days, 2 to 4 days, 2 to 3 days, 3 to 14 days, 3 to 12 days, 3 to 10 days, 3 to 9 days, 3 to 8 days, 3 to 7 days, 3 to 6 days, 3 to 5 days, 3 to 4 days, 4 to 14 days, 4 to 12 days, 4 to 10 days, 4 to 9 days, 4 to 8 days, 4 to 7 days, 4 to 6 days, 4 to 5 days, 5 to 14 days, 5 to 12 days, 5 to 10 days, 5 to 9 days, 5 to 8 days, 5 to 7 days, 5 to 6 days, 6 to 14 days, 6 to 12 days, 6 to 10 days, 6 to 9 days, 6 to 8 days, 6 to 7 days, 7 to 14 days, 7 to 12 days, 7 to 10 days, 7 to 9 days, 7 to 8 days, 8 to 14 days, 8 to 12 days, 8 to 10 days, 8 to 9 days, 9 to 14 days, 9 to 12 days, 9 to 10 days, 10 to 14 days, 10 to 12 days, or 12 to 14 days). In some embodiments, the lifespan of a population of genetically engineered enucleated cells can be evaluated by determining the average time at which a portion of the genetically engineered enucleated cell population (e.g., at least 50%, at least 60%, at least 70%, at least 75%, at least 80%, at least 85%, at least 90%, at least 95%, or at least 98% of the population) is determined to be dead. Cell death can be determined by any method known in the art. In some embodiments, the viability of genetically engineered enucleated cells, e.g., at one or more time points, can be evaluated by determining whether morphometric or functional parameters are intact (e.g., by trypan-blue dye exclusion, evaluating for intact cell membranes, evaluating adhesion to plastics (e.g., in adherent enucleated cells), evaluating genetically engineered enucleated cell migration, negative staining with apoptotic markers, and the like). In some embodiments, the life span of a genetically engineered enucleated cell may be related to the life span of the cell from which it was obtained.

In some embodiments, a genetically engineered enucleated cell has been altered from its native state by depleting the enucleated cell of immune recognition molecules. For example, these immune recognition molecules can be HLA antigens, proteoglycans, sugar moieties, embryonic antigens, or combinations thereof. In some embodiments, the enucleated cell is genetically engineered to express at least one exogenous protein. In some embodiments, the exogenous protein is a cell surface protein. In some embodiments, the exogenous protein is an immune evasion molecule. An immune evasion molecule can be a molecule expressed by a cell, which allows the cell to avoid the innate immune system and to evade immune responses. In some embodiments, an immune evasion molecule is a cytokine, IL-10, CD47, HLA-E/G, PD-1, LAG-3, CTLA-4, or combinations thereof. In some embodiments, the exogenous protein is an immune activating protein. In some embodiments, an immune activating protein is a cytokine, IL-12, calreticulin, phosphatidylysine, phagocytosis prey-binding domain, annexin 1, OX40/OC40L, 4-1BB, B7 family members, or combinations thereof.

### Methods of Making Enucleated Cells

In some embodiments of any of the methods and compositions described herein, a nucleated cell (e.g., an eukaryotic cell, a mammalian cell (e.g., a human cell, a canine cell, a feline cell, an equine cell, a porcine cell, a primate cell, a rodent cell (e.g., a mouse cell, a guinea pig cell, a hamster cell, or a mouse cell), an immune cell, or any nucleated cell described herein), is treated with cytochalasin to soften the cortical actin cytoskeleton. The nucleus is then physically extracted from the cell body by high-speed centrifugation in gradients of Ficoll to generate an enucleated cell. In some embodiments, the nucleus is removed by density gradient centrifugation. As used herein, the term “enucleated cell” can refer to a previously nucleated cell (e.g., any cell described herein) that consists of the inner mass of a cell and the cell organelles. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the nucleated cell is a primary cell. In some embodiments, the nucleated cell is an immune cell (e.g., a T cell, a B cell, a macrophage, a natural killer cell, a neutrophil, a mast cell, a basophil, a dendritic cell, a monocyte, a myeloid-derived suppressor cell, an eosinophil. In some embodiments, the nucleated cell is a phagocyte or a leukocyte. In some embodiments, the nucleated cell is a stem cell (e.g., an adult stem cell, an embryonic stem cell, an inducible pluripotent stem cell (iPS)). In some embodiments, the nucleated cell is a progenitor cell. In some embodiments, the nucleated cell is a cell line. In some embodiments, the nucleated cell is a suspension cell. In some embodiments, the nucleated cell is an adherent cell. In some embodiments, the nucleated cell is a cell that has been immortalized by expression of an oncogene. In some embodiments, the nucleated cell is immortalized by the expression of human telomerase reverse transcriptase (hTERT). In some embodiments, the nucleated cell is a mesenchymal stromal cell (MSC). In some embodiments, the nucleated cell is an hTERT-immortalized adipose-derived MSC (hTERT-MSC). In some embodiments, the nucleated cell is a patient derived cell (e.g., an autologous patient-derived cell, or an allogenic patient-derived cell).

Methods of culturing a cell (e.g., any of the cells described herein) are well known in the art. Cells can be maintained in vitro under conditions that favor growth, proliferation, viability, and differentiation. In some embodiments, the nucleated cells (e.g., MSCs) are cultured in 3D-hanging drops (e.g., 3D MSCs) then enucleated to generate 3D enucleated cells.

In some embodiments of any of the compositions and methods provided herein, the enucleated cell is frozen for later use. Various methods of freezing cells are known in the art, including, but not limited to, the use of a serum (e.g., Fetal Bovine Serum) and dimethyl sulfoxide (DMSO). In some embodiments of any of the compositions and methods provided herein, the enucleated cell is thawed prior to use.

Methods of Introducing a Biomolecule into a Enucleated Cell

Various methods are known in the art that can be used to introduce a biomolecule (e.g., a RNA molecule (e.g., mRNA, miRNA, siRNA, shRNA, lncRNA), a DNA molecule (e.g., a plasmid), a protein, a peptide into an enucleated cell. Non-limiting examples of methods that can be used to introduce a biomolecule into an enucleated cell include: liposome mediated transfer, an adenovirus, an adeno-associated virus, a herpes virus, a retroviral based vector, a lentiviral vector, electroporation, microinjection, lipofection, transfection, calcium phosphate transfection, dendrimer-based transfection, cationic polymer transfection, cell squeezing, sonoporation, optical transfection, impalection, hydrodynamic delivery, magnetofection, nanoparticle transfection, or combinations thereof. In some embodiments of any of the compositions and methods provided herein, a therapeutic agent, a virus, an antibody, or a nanoparticle is introduced into the enucleated cells.

### Immune Evasion

As used herein, the terms “immune evasion” or “to evade immune recognition” refer to a fundamental process in tumor formation and progression. During tumor development, a chronic inflammatory microenvironment reduces the anti-tumoral immune response and favors the escape of tumor from immune elimination. Inflammatory immune cells include tumor-associated macrophages (TAMs), cytotoxic T (CD8) lymphocytes (CTLs), Th (CD4) lymphocytes, natural killer (NK) cells, regulatory T (Treg) cells and myeloid-derived suppressor cells (MDSCs). Among them, Treg cells, MDSCs and macrophages are mainly involved in the immunosuppressive action of key molecules, such as transforming growth factor beta (TGF-β), prostaglandin E2, indoleamine 2, 3-dioxygenase and interleukin-10 (IL-10). Several growth factors, namely TGF-β, insulin-like growth factor 2 (IGF-2) and vascular endothelial growth factor (VEGF), cytokines (e.g., IL-1, IL-4, IL-6, IL-8, IL-10) and tumor-necrosis factor alpha, chemokines (e.g., chemokine (C—X—C motif) ligand 1 and C—C motif chemokine receptor 7) have been reported to be closely involved in tumor progression, invasion and immune evasion.

Further, immune evasion occurs through the selection of immune evasion molecules (e.g., tumor variants) that become resistant to an immune attack primarily mediated by T cells and natural killer (NK) cells. For example, an immune evasion molecule can be a cytokine (e.g., IL-1, IL-4, IL-6, IL-8, IL-10), TGF-0, IGF-2, VEGF, TNF-alpha, CD47, HLA-E, HLA-G, HLA-E/G, PD-1, PD-L1, TIGIT, CD112R, CTLA-4, chemokines (e.g., chemokine ligand 1, C—C motif chemokine receptor 7), or any combinations thereof. In some embodiments, an immune evasion molecule can be an NK inhibitor receptor (e.g., HLA-class I-specific inhibitory receptors, e.g., killer cell immunoglobulin-like receptor (KIR), NKG2A, or lymphocyte activation gene-3 (LAG-3)).

The generation of bioengineered enucleated cells offer to improve therapeutic functions and produce cell-like entities that are controllable and safe. The bioengineered enucleated cells designed to evade recognition by the immune system and further therapeutic use offer several benefits over previous cell-based therapeutics, including, e.g., safety, defined lifespan, no risk of nuclear-encoded gene transfer to host, and effective delivery of therapeutic cargo. In some embodiments, enucleated cells can be genetically engineered to express a cytokine (e.g., IL-1, IL-4, IL-6, IL-8, IL-10), TGF-β, IGF-2, VEGF, TNF-alpha, CD47, HLA-E, HLA-G, HLA-E/G, PD-1, PD-L1, TIGIT, CD112R, CTLA-4, chemokines (e.g., chemokine ligand 1, C—C motif chemokine receptor 7), or any combinations thereof. In some embodiments, enucleated cells can be genetically engineered to express an NK inhibitor receptor (e.g., HLA-class I-specific inhibitory receptors, e.g., killer cell immunoglobulin-like receptor (KIR), NKG2A, or lymphocyte activation gene-3 (LAG-3)).

In some embodiments, a method of governing immune recognition in a subject includes, administering to the subject an enucleated cell, wherein the enucleated cell is genetically engineered to evade recognition by the immune system. In some embodiments, the enucleated cell is genetically engineered to deplete the enucleated cell of immune recognition molecules. In some embodiments, the immune recognition molecules include, but are not limited to, HLA antigens, proteoglycans, sugar moieties, embryonic antigens, or combinations thereof. In some embodiments, the enucleated cell is genetically engineered to express at least one exogenous protein. In some embodiments, the exogenous protein is a cell surface protein. In some embodiments, the exogenous protein is an immune evasion molecule. In some embodiments, the exogenous protein includes a cytokine (e.g., IL-1, IL-4, IL-6, IL-8, IL-10), TGF-β, IGF-2, VEGF, TNF-alpha, CD47, HLA-E, HLA-G, HLA-E/G, PD-1, PD-L1, TIGIT, CD112R, CTLA-4, chemokines (e.g., chemokine ligand 1, C—C motif chemokine receptor 7), or any combinations thereof. In some embodiments, the exogenous protein includes an NK inhibitor receptor (e.g., HLA-class I-specific inhibitory receptors, e.g., killer cell immunoglobulin-like receptor (KIR), NKG2A, or lymphocyte activation gene-3 (LAG-3)).

### Immune Activation

As used herein, the term “immune activation” refers to the transition of leukocytes (e.g., macrophages, neutrophils, NK cells) and other cell types involved in the immune system. Activation of the immune system is a pathologically appropriate response to invading pathogens. Immune activation provides a beneficial role in control and clearance of invading pathogens. Also, surveillance and activity of the immune system contributes of control and suppression of pathogen replication and spread. Further, cancer immunotherapy uses the immune system and its components to mount an anti-tumor response through immune activation. Immune activation proteins can include, but are not limited to, a cytokine, IL-12, calreticulin, phosphatidylysine, phagocytosis prey-binding domain, annexin 1, OX40/OC40L, 4-1BB, CD70, GITRL, LIGHT, CD30L, B7 family members, or combinations thereof.

The bioengineered enucleated cells designed for immune activation and further therapeutic use offer several benefits over previous cell-based therapeutics, and further allow better understanding of the activation and regulation of innate immune signaling in the immune response to pathogens and cancer. In some embodiments, enucleated cells can be genetically engineered to express a cytokine, IL-12, calreticulin, phosphatidylysine, phagocytosis prey-binding domain, annexin 1, OX40/OC40L, 4-1BB, CD70, GITRL, LIGHT, CD30L, B7 family members, or combinations thereof.

In some embodiments, a method of governing immune activation in a subject includes, administering to the subject an enucleated cell, wherein the enucleated cell is genetically engineered to activate the immune system. In some embodiments, the enucleated cell activates an immune response in the subject. In some embodiments, the enucleated cell is genetically engineered to express at least one exogenous protein. In some embodiments, the exogenous protein is a cell surface protein. In some embodiments, the exogenous protein is an immune activating protein. In some embodiments, the exogenous protein comprises a cytokine, IL-12, calreticulin, phosphatidylysine, phagocytosis prey-binding domain, annexin 1, OX40/OC40L, 4-1BB, B7 family members, or combinations thereof

### Compositions, Including Combination Therapies

In some embodiments, the present disclosure provides pharmaceutical compositions that include an enucleated cell and a pharmaceutically acceptable carrier. In some embodiments, the composition can be used as a disease-homing vehicle to deliver clinically relevant cargos/payloads to treat various diseases. In some embodiments, the composition can be used for treating or diagnosing a disease.

In some embodiments, the composition includes one or more enucleated cells genetically engineered to express at least one exogenous protein. In some embodiments, the exogenous protein is a cell surface protein. In some embodiments, the exogenous protein is an immune evasion molecule. In some embodiments, the composition includes an immune evasion molecule, for example, a cytokine (e.g., IL-1, IL-4, IL-6, IL-8, IL-10), CD47, HLA-E/G, PD-1, LAG-3, CTLA-4, PD-L1, TIGIT, CD112R, and NK inhibitor receptors, such as HLA-class I-specific inhibitory receptors (e.g., killer cell immunoglobin-like receptor (KIR), NKG2A, and lymphocyte activation gene-3 (LAG-3)), or combinations thereof. In some embodiments, the exogenous protein is an immune activating protein. In some embodiments, the composition includes an immune activating protein, for example, a cytokine, IL-12, calreticulin, phosphatidylysine, phagocytosis prey-binding domain, annexin 1, OX40/OC40L, 4-1BB, B7 family members, or combinations thereof.

In some embodiments, a pharmaceutical composition can include a buffer, a diluent, a solubilizer, an emulsifier, a preservative, an adjuvant, an excipient, or any combination thereof. In some embodiments, a composition can be formulated for parenteral administration. For example, a pharmaceutical composition provided herein may be provided in a sterile injectable form (e.g., a form that is suitable for subcutaneous injection or intravenous infusion). For example, in some embodiments, a pharmaceutical composition is provided in a liquid dosage form that is suitable for injection.

In some embodiments, the pharmaceutical composition is formulated with a pharmaceutically acceptable parenteral vehicle. For example, such vehicles can include, but are not limited to, water, saline, Ringer's solution, dextrose solution, and human serum albumin. Liposomes and nonaqueous vehicles such as fixed oils can also be used. In some embodiments, a formulation is sterilized by known or suitable techniques. In some embodiments, a pharmaceutical composition may additionally comprise a pharmaceutically acceptable excipient, which can include any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

In some embodiments of any of the methods provided herein, the pharmaceutical composition is administered with one or more additional therapies (e.g., chemotherapy (e.g., a chemotherapeutic agent (e.g., doxorubicin, paclitaxel, cyclophosphamide), cell-based therapy, radiation therapy, immunotherapy, a small molecule, an inhibitory nucleic acid (e.g., antisense RNA, antisense DNA, miRNA, siRNA, lncRNA), an exosome-based therapy, gene therapy or surgery). In some embodiments, the one or more additional therapies include immune checkpoint blockade, wherein immune checkpoint inhibitors are administered. In some embodiments, the immune checkpoint inhibitors can include, but are not limited to, PD-1 inhibitors, PD-L1 inhibitors, TIM-3 inhibitors, LAG-3 inhibitors, TIGIT inhibitors, CD47 inhibitors, B7 inhibitors, CD137 inhibitors, or CTLA-4 inhibitors. In some embodiments, the immune checkpoint inhibitor can include a PD-1 inhibitor including, but not limited to, Pembrolizumab, Nivolumab, or Cemiplimab. In some embodiments, the immune checkpoint inhibitor can include a PD-L1 inhibitor including, but not limited to, Atezolizumab, Avelumab, or Durvalumab. In some embodiments, the immune checkpoint inhibitor can include a LAG-3 inhibitor including, but not limited to, relatlimab. In some embodiments, the immune checkpoint inhibitor can include a CTLA-4 inhibitor including, but not limited to, Ipilimumab. In some embodiments, the composition including the enucleated cell is administered simultaneously with the one or more additional therapies. In some embodiments, the composition including the enucleated cell is administered separately from the one or more additional therapies.

In some embodiments of any of the compositions provided herein, the composition further includes one or more additional therapies (e.g., chemotherapy (e.g., a chemotherapeutic agent (e.g., doxorubicin, paclitaxel, cyclophosphamide), cell-based therapy, radiation therapy, immunotherapy, a small molecule, an inhibitory nucleic acid (e.g., antisense RNA, antisense DNA, miRNA, siRNA, lncRNA) or surgery). In some embodiments, the one or more additional therapies include immune checkpoint blockade, wherein immune checkpoint inhibitors are administered. In some embodiments, the immune checkpoint inhibitors can include, but are not limited to, PD-1 inhibitors, PD-L1 inhibitors, TIM-3 inhibitors, LAG-3 inhibitors, TIGIT inhibitors, CD47 inhibitors, B7 inhibitors, CD137 inhibitors, or CTLA-4 inhibitors. In some embodiments, the composition can further include a PD-1 inhibitor including, but not limited to, Pembrolizumab, Nivolumab, or Cemiplimab. In some embodiments, the composition can further include a PD-L1 inhibitor including, but not limited to, Atezolizumab, Avelumab, or Durvalumab. In some embodiments, the composition can further include a LAG-3 inhibitor including, but not limited to, relatlimab. In some embodiments, the composition can further include a CTLA-4 inhibitor including, but not limited to, Ipilimumab. In some embodiments, a composition can also contain one or more additional therapeutically active substances.

In some embodiments, a pharmaceutical composition can include one population of enucleated cells, wherein substantially all the enucleated cells are genetically engineered to express the same molecule, such as the same exogenous DNA molecule, exogenous RNA molecule, exogenous polypeptide, or exogenous protein. In some embodiments, the one population of enucleated cells is engineered to express one biomolecule (e.g., cargo). In some embodiments, the one population of enucleated cells is engineered to express two or more biomolecules (e.g., two biomolecules, three biomolecules, four biomolecules, or five biomolecules). In some embodiments, the one population of enucleated cells is engineered to express two biomolecules, wherein the two exogenous molecules introduced to express the two biomolecules could be the same type of molecule. For example, the one population of enucleated cells engineered to express two biomolecules could be loaded with two different exogenous DNA molecules. In embodiments wherein the one population of enucleated cells is engineered to express two or more biomolecules, each exogenous molecule introduced to express the payload could be different. For example, in one population of enucleated cells engineered to express two biomolecules, wherein one molecule expressing one biomolecule could be an exogenous DNA molecule, and a second molecule expressing a second biomolecule could be an exogenous RNA molecule.

In some embodiments, a pharmaceutical composition can include different populations of enucleated cells, wherein each population is engineered to express a different exogenous molecule (e.g., an exogenous DNA molecule, an exogenous RNA molecule, an exogenous polypeptide, or an exogenous protein, or combinations thereof). For example, a pharmaceutical composition can include one population of enucleated cells genetically engineered to express a cytokine (e.g., IL-1, IL-4, IL-6, IL-8, IL-10), and a second population of enucleated cells genetically engineered to express a checkpoint inhibitor (PD-1 inhibitors, PD-L1 inhibitors, TIM-3 inhibitors, LAG-3 inhibitors, TIGIT inhibitors, CD47 inhibitors, B7 inhibitors, CD137 inhibitors, or CTLA-4 inhibitors). Additional examples include, but are not limited to a pharmaceutical composition including one population of enucleated cells genetically engineered to express IL-12, and a second population of enucleated cells genetically engineered to express a PD-1 inhibitor. An additional example includes, a pharmaceutical composition including one population of enucleated cells genetically engineered to express CXCR4, a second population of enucleated cells genetically engineered to express CCR2, and a third population of enucleated cells genetically engineered to express PGSL-1/FUT-7. In some embodiments, a pharmaceutical composition can include different populations of enucleated cells, wherein one population of enucleated cells is engineered to express one biomolecule, and a second population of enucleated cells is engineered to express two or more biomolecules. In some embodiments, a pharmaceutical composition can include two populations of enucleated cells, wherein each population is engineered to express two or more biomolecules.

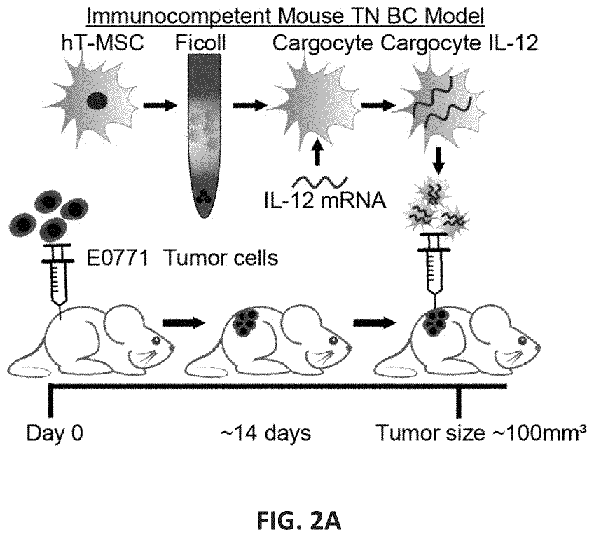

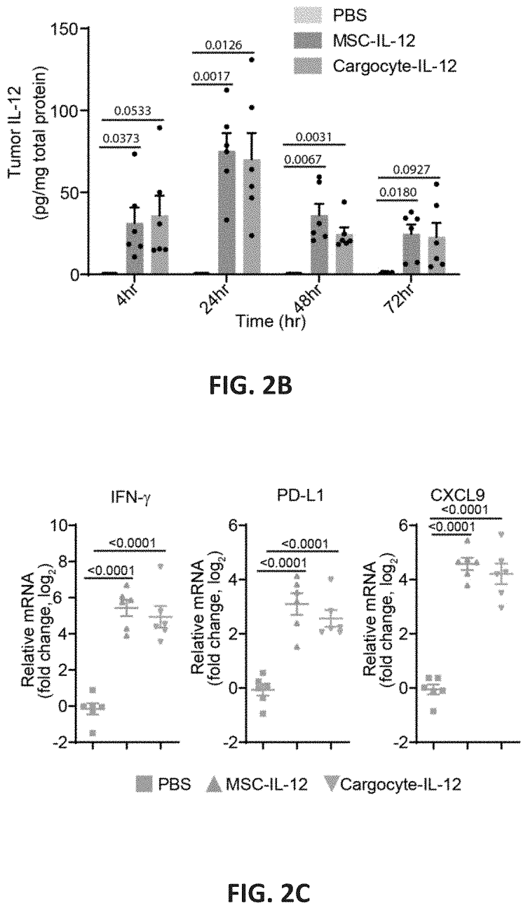

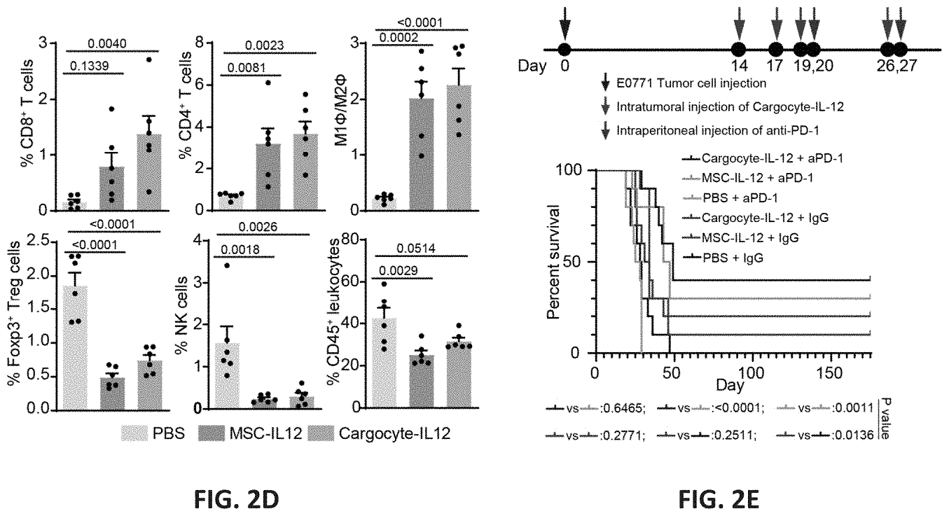

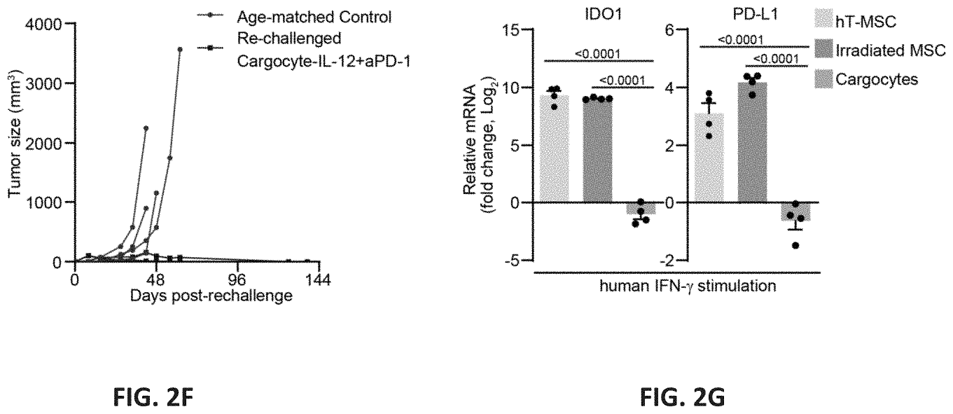

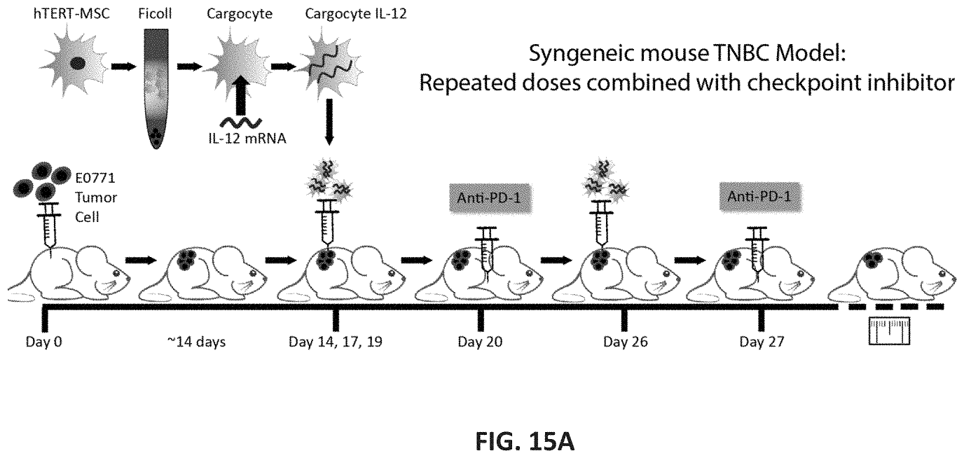

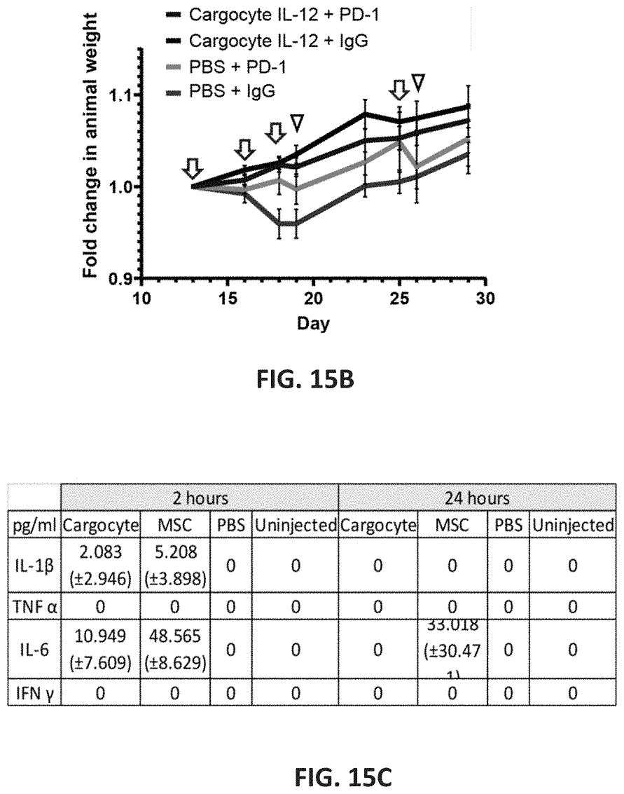

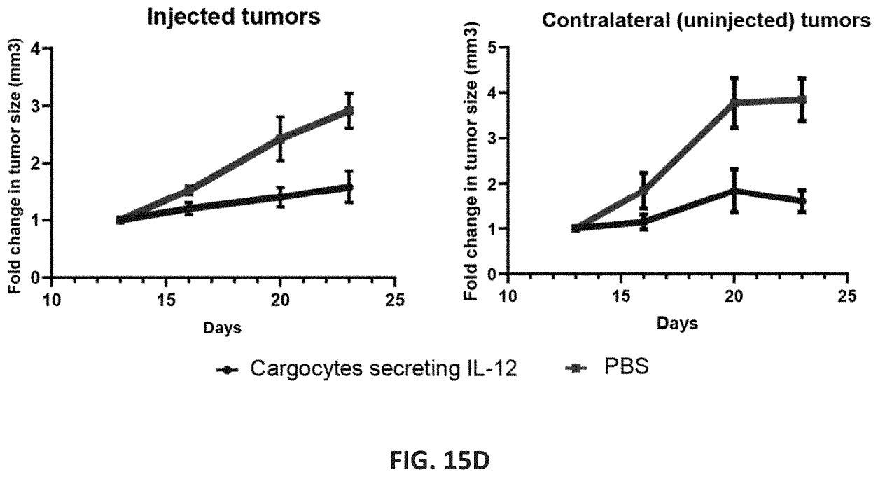

In some embodiments, combination therapies, whether the composition includes one or more population(s) of enucleated cells engineered to express one or more biomolecules, or wherein the composition includes one or more population(s) of enucleated cells engineered to express one or more biomolecules and further includes a separate therapeutic, exhibits synergism as a therapeutic. Synergism, in some contexts, can mean that the combination of biomolecules and/or therapies produces a more beneficial effect (e.g., stronger, longer lasting, better tolerated, etc.) than expected based on the responses to each biomolecule and/or therapy alone. In some embodiments, a combination therapy, wherein an enucleated cell and a checkpoint inhibitor is administered, can produce synergistic effects of treating a disease. For example, when enucleated cells genetically engineered to express IL-12 are administered with the PD-1 checkpoint inhibitor, it has been shown to significantly reduce tumor growth and improve survival in a mouse model (e.g., FIGS. 2E-2F and FIGS. 15A-15D).

### Methods of Use as Disease Diagnostics

The present disclosure provides methods for the use of enucleated cells (natural or engineered) to enhance and/or transfer biomolecules (secreted, intracellular, and natural and inducible) including, but not limited to, DNA/genes, RNA (mRNA, shRNA, siRNA, miRNA), nanoparticles, peptides, proteins, and plasmids, bacteria, viruses, small molecule drugs, ions, cytokines, growth factors, and hormones.

Enucleated cells (e.g., cargocytes) are smaller in diameter while lacking rigid nuclei and are expected to pass through small constrictions such as capillaries or interstitial spaces more effectively than nucleated parental cells. For example, enucleated cells have been shown to pass through microvasculature better than nucleated parental cells, therefore better facilitating in vivo homing to damaged or inflamed tissue.

In some embodiments, the enucleated cell is genetically engineered to express an “inflammation homing receptor”, wherein “inflammation homing receptor” herein, refers to an adhesion molecule on leukocytes that binds to endothelial cells in blood vessels. Inflammation homing receptors are used by white blood cells to guide them to sites of tissue inflammation in the body. These diverse tissue-specific adhesion molecules on lymphocytes (e.g., homing receptors) and on endothelial cells (e.g., vascular addressins) contribute to the development of specialized immune responses. In some embodiments, an inflammation homing receptor is an α4β7, VCAM-1, CD34, GLYCAM-1, LFA-1, CD44, and combinations thereof.

In some embodiments, the enucleated cell is genetically engineered to express a “firefly luciferase”, wherein “firefly luciferase” herein, refers to a light-emitting enzyme and bioluminescent reporter for studying gene regulation and function. It is a very sensitive genetic reporter due to the absence of endogenous luciferase activity in mammalian cells or tissues. Firefly luciferase is a 62,000 Dalton protein, which is active as a monomer and does not require subsequent processing for its activity. The enzyme catalyzes ATP-dependent D-luciferin oxidation to oxyluciferin, producing light emission centered at 560 nm. Light emitted from the reaction is directly proportional to the number of luciferase enzyme molecules.

In some embodiments, the enucleated cell is genetically engineered to express only an inflammation homing receptor. In some embodiments, the enucleated cell is genetically engineered to express only firefly luciferase. In some embodiments, the enucleated cell is genetically engineered to express both an inflammation homing receptor and firefly luciferase.

In some embodiments, an enucleated cell is used to identify the presence of a disease condition in a subject by administering to the subject an enucleated cell genetically engineered to express at least one of an exogenous DNA molecule, an exogenous RNA molecule, an exogenous protein, or an exogenous peptide, wherein the genetically engineered enucleated cell identifies the presence or location of a disease condition. In some embodiments, the exogenous protein is an inflammation homing receptor. In some embodiments, the inflammation homing receptor directs the enucleated cell to damaged and/or inflamed tissue.

### Methods of Treatment

The present methods include the use of enucleated cells for treating or diagnosing a disease (e.g., a cancer (e.g., multiple myeloma, glioblastoma, lymphoma, a solid cancer, a leukemia), an infection (e.g., viral infections, such as but not limited to, human immunodeficiency virus (HIV)-infection, Severe Acute Respiratory Syndrome or COVID-19 infection (coronavirus infection), parasitic infection, such as but not limited to, Chagas disease, or bacterial infection, such as but not limited to, tuberculosis), a neurological disease (e.g., Parkinson's Disease, Huntington's Disease, Alzheimer's Disease) an autoimmune disease (e.g., diabetes, Crohn's disease, multiple sclerosis, sickle cell anemia), a cardiovascular disease (e.g., acute myocardial infarction, heart failure, refractory angina), a ophthalmologic disease, a skeletal disease, a metabolic disease (e.g., phenylketonuria, glycogen storage deficiency type 1A, Gaucher disease)) in a subject. In some embodiments, the subject is in need of, or has been determined to be in need of, such an enucleated cell treatment. As used herein, the term “subject” refers to any mammal. In some embodiments, the subject may be a rodent (e.g., a mouse, a rat, a hamster, a guinea pig), a canine (e.g., a dog), a feline (e.g., a cat), an equine (e.g., a horse), an ovine, a bovine, a porcine, a primate, e.g., a simian (e.g., a monkey), an ape (e.g., a gorilla, a chimpanzee, an orangutan, a gibbon), or a human. As used herein, treating includes “prophylactic treatment” which means reducing the incidence of or preventing (or reducing risk of) a sign or symptom of a disease in a subject at risk for the disease, and “therapeutic treatment”, which means reducing signs or symptoms of a disease, reducing progression of a disease, reducing severity of a disease, re-occurrence in a subject diagnosed with the disease. As used herein, the term “treat” means to ameliorate at least one clinical parameter of the disease.

As used herein, the term “administration,” “administering” and variants thereof means introducing a composition or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. A suitable route of administration allows the composition or the agent to perform its intended function. For example, if a suitable route is intravenous, the composition is administered by introducing the composition or agent into a vein of the subject. Administration can be carried out by any suitable route.

In some embodiments of any of the methods provided herein, the composition is administered at least once (e.g., 2, 3, 4, 5, 6, 7, 8, 9, 10, 11, 12, 13, 14, 15, 16, 17, 18, 19, 20, 30, 40, 50, 60, 70 80, 90, 100 times) during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year).

In some embodiments, a method of treating a disease in a subject includes administering to the subject a therapeutically effective amount of a composition comprising an enucleated cell genetically engineered to express at least one of an exogenous DNA molecule, an exogenous RNA molecule, an exogenous protein, or an exogenous peptide. In some embodiments, the composition further comprises a therapeutic agent. In some embodiments, the therapeutic agent comprises at least one of a small RNA, a small molecule drug, a peptide, a virus, or combinations thereof. In some embodiments, the therapeutic agent comprises a chemotherapeutic agent.

A number of embodiments have been described. Nevertheless, it will be understood that various modifications may be made without departing form the spirit and scope of the invention.

### EXEMPLARY EMBODIMENTS

Embodiment 1. A method of treating a disease in a subject, the method comprising: administering to the subject a therapeutically effective amount of a composition comprising an enucleated cell genetically engineered to express at least one of an exogenous DNA molecule, an exogenous RNA molecule, an exogenous protein, or an exogenous peptide.
Embodiment 2. The method of embodiment 1, wherein the enucleated cell is engineered to express two or more exogenous DNA molecules, exogenous RNA molecules, exogenous proteins, or exogenous peptides, or combinations thereof.
Embodiment 3. The method of embodiment 1, wherein the enucleated cell is engineered to express three or more exogenous DNA molecules, exogenous RNA molecules, exogenous proteins, or exogenous peptides, or combinations thereof.
Embodiment 4. The method of embodiment 1, wherein the enucleated cell is engineered to express four or more exogenous DNA molecules, exogenous RNA molecules, exogenous proteins, or exogenous peptides, or combinations thereof.
Embodiment 5. The method of embodiment 1, wherein the enucleated cell is derived from a natural killer (NK) cell, a macrophage, a neutrophil, a fibroblast, and adult stem cell, a mesenchymal stromal cell (MSC), an inducible pluripotent stem cell, or combinations thereof.
Embodiment 6. The method of embodiment 1, wherein the enucleated cell is derived from a mesenchymal stromal cell (MSC).
Embodiment 7. The method of embodiment 1, wherein the exogenous DNA molecule comprises single-stranded DNA, double-stranded DNA, an oligonucleotide, a plasmid, a bacterial DNA molecule, a DNA virus, or combinations thereof.
Embodiment 8. The method of embodiment 1, wherein the exogenous RNA molecule comprises messenger RNA (mRNA), small interfering RNA (siRNA), microRNA (miRNA), short hairpin RNA (shRNA), a RNA virus, or combinations thereof.
Embodiment 9. The method of embodiment 1, wherein the exogenous protein comprises a cytokine, a growth factor, a hormone, an antibody, an enzyme, or combinations thereof.
Embodiment 10. The method of embodiment 1, wherein the enucleated cell of the composition is selected using fluorescence activated cell sorting (FACS).
Embodiment 11. The method of embodiments 1-10, wherein the enucleated cell further comprises a therapeutic agent.
Embodiment 12. The method of embodiment 11, wherein the therapeutic agent comprises at least one of a small RNA, a small molecule drug, a peptide, a virus, or combinations thereof
Embodiments 13. The method of embodiment 11, wherein the therapeutic agent comprises a chemotherapeutic agent.
Embodiment 14. The method of embodiments 1-13, wherein the administering comprises intravenous administration, subcutaneous administration, intraperitoneal administration, rectal administration, oral administration, or combinations thereof.
Embodiment 15. The method of embodiments 1-14, wherein the composition exhibits minimal accumulation in non-target tissues.
Embodiment 16. The method of embodiments 1-15, wherein the administering is within the site of disease.
Embodiment 17. The method of embodiment 1-16, wherein the disease comprises inflammation, an infection, cancer, a neurological disease, an autoimmune disease, a cardiovascular disease, an ophthalmologic disease, a skeletal disease, a metabolic disease, or combinations thereof.
Embodiment 18. The method of embodiments 1-17, wherein the disease comprises inflammation.
Embodiment 19. The method of embodiments 1-18, wherein the disease comprises cancer.
Embodiment 20. The method of embodiment 19, wherein the cancer comprises multiple myeloma, glioblastoma, lymphoma, leukemia, mesothelioma, sarcoma, breast cancer, prostate cancer, ovarian cancer, pancreatic cancer, colon cancer, or combinations thereof.
Embodiment 21. The method of embodiments 1-20, wherein the administering comprises intratumoral administration.
Embodiment 22. The method of embodiments 1-21, wherein the method inhibits cancer progression.
Embodiment 23. The method of embodiments 1-22, wherein the method reduces tumor growth.
Embodiment 24. The method of embodiments 1-23, wherein the method produces complete tumor regression.
Embodiment 25. The method of embodiments 1-24, wherein the method improves subject likelihood of survival.
Embodiment 26. The method of embodiments 1-25, wherein the method produces a systemic anti-tumor immune response.
Embodiment 27. The method of embodiments 1-26, wherein the composition of enucleated cells is over 90% pure.
Embodiment 28. The method of embodiments 1-27, wherein the composition is over 95% pure.
Embodiment 29. The method of embodiments 1-28, wherein the composition is over 98% pure.
Embodiment 30. The method of embodiments 1-29, wherein the composition is over 99% pure.
Embodiment 31. A method of genetically engineering an enucleated cell, the method comprising:
enucleating a nucleated cell; and
introducing into the enucleated cell an exogenous DNA molecule, an exogenous RNA molecule, an exogenous protein, an exogenous peptide and/or a therapeutic agent, wherein the genetically engineered enucleated cell retains functional translation and secretory machinery of a parental cell in vivo.
Embodiment 32. The method of embodiment 31, wherein the introducing step occurs before enucleation of the nucleated cell.
Embodiment 33. The method of embodiment 31, wherein the introducing step occurs after the enucleation of the nucleated cell.
Embodiment 34. The method of embodiments 31-33, wherein the enucleating step is over 95% efficient.
Embodiment 35. The method of embodiments 31-34, wherein the method has at least an 80% recovery rate.
Embodiment 36. The method of embodiments 31-35, wherein the method has at least an 85% recovery rate.
Embodiment 37. The method of embodiments 31-36, wherein the introducing step comprises viral transduction.
Embodiment 38. The method of embodiments 31-37, wherein the introducing step comprises using at least one of liposome mediated transfer, an adenovirus, an adeno-associated virus, a herpes virus, a retroviral based vector, lipofection, a lentiviral vector, or combinations thereof.
Embodiment 39. The method of embodiments 31-38, wherein the exogenous DNA molecule comprises single-stranded DNA, double-stranded DNA, an oligonucleotide, a plasmid, a bacterial DNA molecule, a DNA virus, or combinations thereof.
Embodiment 40. The method of embodiments 31-38, wherein the exogenous RNA molecule comprises messenger RNA (mRNA), small interfering RNA (siRNA), microRNA (miRNA), short hairpin RNA (shRNA), a RNA virus, or combinations thereof.
Embodiment 41. The method of embodiments 31-38, wherein the exogenous protein comprises a cytokine, a growth factor, a hormone, an antibody, an enzyme, or combinations thereof.
Embodiment 42. The method of embodiments 31-41, further comprising cryopreserving the genetically engineered enucleated cell.
Embodiment 43. The method of embodiment 42, wherein the genetically engineered enucleated cell is more likely to recover from cryopreservation compared to a parental cell.
Embodiment 44. A genetically engineered enucleated cell produced by introducing into an enucleated cell a least one of an exogenous DNA molecule, an exogenous RNA molecule, an exogenous protein, an exogenous peptide, or a therapeutic agent.
Embodiment 45. The genetically engineered enucleated cell of embodiment 44, the enucleated cell is engineered to express two or more exogenous DNA molecules, exogenous RNA molecules, exogenous proteins, or exogenous peptides, or combinations thereof.
Embodiment 46. The genetically engineered enucleated cell of embodiment 44, the enucleated cell is engineered to express three or more exogenous DNA molecules, exogenous RNA molecules, exogenous proteins, or exogenous peptides, or combinations thereof.
Embodiment 47. The genetically engineered enucleated cell of embodiment 44, the enucleated cell is engineered to express four or more exogenous DNA molecules, exogenous RNA molecules, exogenous proteins, or exogenous peptides, or combinations thereof.
Embodiment 48. The genetically engineered enucleated cell of embodiment 44, wherein the enucleated cell is derived from a mesenchymal stromal cell (MSC).
Embodiment 49. The genetically engineered enucleated cell of embodiment 48, wherein the enucleated cell is derived from an hTERT-immortalized adipose-derived MSC (hT-MSC).
Embodiment 50. The genetically engineered enucleated cell of embodiment 49, wherein the enucleated cell secretes similar extracellular vesicles (EVs) as compared to a parental hT-MSC cell.
Embodiment 51. The genetically engineered enucleated cell of embodiment 44, wherein the introducing step comprises viral transduction.
Embodiment 52. The genetically engineered enucleated cell of embodiment 44, wherein the introducing step comprises at least one of liposome mediated transfer, adenovirus, adeno-associated virus, herpes virus, a retroviral based vector, lipofection, a lentiviral vector, or combinations thereof.
Embodiment 53. The genetically engineered enucleated cell of embodiment 44, which exhibits substantially the same cell structure as a parental cell.
Embodiment 54. The genetically engineered enucleated cell of embodiment 53, wherein the genetically enucleated cell contains functional subcellular organelles.
Embodiment 55. The genetically engineered enucleated cell of embodiment 54, wherein the functional subcellular organelles comprise at least one of the Golgi, endoplasmic reticulum, mitochondria, lysosomes, endosomes, ribosomes, or combinations thereof.
Embodiment 56. The genetically engineered enucleated cell of embodiment 44, which exhibits substantially the same cell function as a parental cell.
Embodiment 57. The genetically engineered enucleated cell of embodiment 56, wherein the genetically enucleated cell has substantially the same zeta potential than that of a parental cell.
Embodiment 58. The genetically engineered enucleated cell of embodiment 56, wherein the genetically enucleated cell contains functional membrane receptors.
Embodiment 59. The genetically engineered enucleated cell of embodiment 56, wherein the genetically enucleated cell contains functional migration and invasion machinery.
Embodiment 60. The genetically engineered enucleated cell of embodiment 56, wherein the genetically enucleated cell can actively produce and secrete substantially the same extracellular vesicles as those produced by a parental cell.
Embodiment 61. The genetically engineered enucleated cell of embodiments 44-60, wherein the genetically enucleated cell produces therapeutic bioactive proteins in vivo.
Embodiment 62. The genetically engineered enucleated cell of embodiments 44-61, wherein the genetically enucleated cell is engineered to express a cell surface protein.
Embodiment 63. The genetically engineered enucleated cell of embodiment 44, wherein the cell surface protein comprises CXCR4, CCR2, PSGL-1, CD44, CD90, CD105, CD106, CD166, Stro-1, or combinations thereof.
Embodiment 64. The genetically engineered enucleated cell of embodiments 44-63, wherein the diameter of the genetically engineered enucleated cell is smaller than that of a parental cell.
Embodiment 65. The genetically engineered enucleated cell of embodiments 44-64, wherein the diameter of the genetically engineered enucleated cell is about 1 micrometers to 100 micrometers.
Embodiment 66. The genetically engineered enucleated cell of embodiments 44-65, which was derived from cells cultured in hanging drop cell culture.
Embodiment 67. The genetically engineered enucleated cell of embodiments 44-66, wherein the genetically enucleated cell is viable for up to 72 hours post-enucleation.
Embodiment 68. The genetically engineered enucleated cell of embodiments 44-67, wherein the genetically enucleated cell retains MSC surface marker protein expression for at least 48 hours.
Embodiment 69. The genetically engineered enucleated cell of embodiments 44-68, wherein the genetically enucleated cell responds to an extracellular signal.
Embodiment 70. The genetically engineered enucleated cell of embodiment 69, wherein the extracellular signal is a chemokine.
Embodiment 71. The genetically engineered enucleated cell of embodiments 44-70, wherein the genetically enucleated cell is capable of chemotaxis.
Embodiment 72. The genetically engineered enucleated cell of embodiments 44-71, wherein the genetically enucleated cell is capable of protein secretion.
Embodiment 73. The genetically engineered enucleated cell of embodiments 44-72, wherein the genetically enucleated cell is capable of homing.
Embodiment 74. The genetically engineered enucleated cell of embodiments 44-73, wherein the genetically enucleated cell is capable of delivering a target product at a target site in vivo.
Embodiment 75. A method of governing immune recognition and/or activation in a subject, the method comprising:
administering to the subject an enucleated cell, wherein the enucleated cell is genetically engineered to evade recognition by the immune system and/or activate the immune system.
Embodiment 76. The method of embodiment 75, wherein the enucleated cell evades immune recognition in the subject.
Embodiment 77. The method of embodiment 76, wherein the enucleated cell is genetically engineered to deplete the enucleated cell of immune recognition molecules.
Embodiment 78. The method of embodiment 77, wherein the immune recognition molecules comprise HLA antigens, proteoglycans, sugar moieties, embryonic antigens, or combinations thereof.
Embodiment 79. The method of embodiment 76, wherein the enucleated cell is genetically engineered to express at least one exogenous protein.
Embodiment 80. The method of embodiment 79, wherein the exogenous protein is a cell surface protein.
Embodiment 81. The method of embodiment 80, wherein the exogenous protein is an immune evasion molecule.
Embodiment 82. The method of embodiments 79-81, wherein the exogenous protein comprises a cytokine, IL-10, CD47, HLA-E/G, PD-1, LAG-3, CTLA-4, or combinations thereof.
Embodiment 83. The method of embodiment 75, wherein the enucleated cell activates an immune response in the subject.
Embodiment 84. The method of embodiment 83, wherein the enucleated cell is genetically engineered to express at least one exogenous protein.
Embodiment 85. The method of embodiment 84, wherein the exogenous protein is a cell surface protein.
Embodiment 86. The method of embodiment 85, wherein the exogenous protein is an immune activating protein.
Embodiment 87. The method of embodiments 84-86, wherein the exogenous protein comprises a cytokine, IL-12, calreticulin, phosphatidylysine, phagocytosis prey-binding domain, annexin 1, OX40/OC40L, 4-1BB, B7 family members, or combinations thereof.
Embodiment 88. The method of embodiments 75-87, further comprising treating a disease in the subject.
Embodiment 89. The method of embodiment 88, wherein the disease comprises an inflammation disorder and/or a cancer.
Embodiment 90. The method of embodiment 89, wherein the cancer comprises multiple myeloma, glioblastoma, lymphoma, leukemia, mesothelioma, sarcoma, breast cancer, prostate cancer, ovarian cancer, pancreatic cancer, colon cancer, or combinations thereof.
Embodiment 91. A method of identifying the presence of a disease condition in a subject, the method comprising:
administering to the subject an enucleated cell genetically engineered to express at least one of an exogenous DNA molecule, an exogenous RNA molecule, an exogenous protein, or an exogenous peptide,
wherein the genetically engineered enucleated cell identifies the presence or location of a disease condition.
Embodiment 92. The method of embodiment 91, wherein the exogenous protein is an inflammation homing receptor.
Embodiment 93. The method of embodiment 92, wherein the inflammation homing receptor directs the enucleated cell to damaged and/or inflamed tissue.
Embodiment 94. The method of embodiment 91-93, wherein the enucleated cell further comprises a firefly luciferase.
Embodiment 95. The method of embodiment 94, wherein the firefly luciferase emits detectable light.
Embodiment 96. The method of embodiment 91-95, wherein the disease comprises inflammation, an infection, cancer, a neurological disease, an autoimmune disease, a cardiovascular disease, an ophthalmologic disease, a skeletal disease, a metabolic disease, or combinations thereof.
Embodiment 97. The method of embodiment 96, wherein the disease comprises a cancer.
Embodiment 98. The method of embodiment 97, wherein the cancer comprises multiple myeloma, glioblastoma, lymphoma, leukemia, mesothelioma, sarcoma, breast cancer, prostate cancer, ovarian cancer, pancreatic cancer, colon cancer, or combinations thereof.

### EXAMPLES

The disclosure is further described in the following examples, which do not limit the scope of the disclosure.

### Example 1—Genetically Engineering Enucleated Cells

First, parental cells (e.g., nucleated cells) were genetically engineered before enucleation of MSCs. G-protein-coupled receptors such as CXCR4 transduce extracellular stimuli into intracellular signals and regulate important cellular functions. The hT-MSCs were engineered with stable CXCR4 expression (MSC^{CXCR4}) via lentivirus infection and drug selection. After enucleation, enucleated cells derived from MSC^{CXCR4} show stable surface expression of CXCR4 by flow cytometry for up to 48 hours, were shown to be viable for up to 72 hours post-enucleation (FIGS. 1B and 1C), and are significantly smaller than hT-MSCs in suspension (FIG. 1G). Importantly, the enucleated cells^{CXCR4} responded and migrated towards chemokine gradients of the cognate ligand SDF-1α in a dose-dependent manner (FIG. 1D), indicating that the membrane-expressed receptor and downstream signaling pathways in enucleated cells^{CXCR4} are fully functional. Next, enucleated cells were genetically engineered post-enucleation by developing a method to efficiently transfect enucleated cells with artificial mRNAs synthesized in vitro. Following transfection with GFP mRNA, epi-fluorescent images and flow cytometry analyses show that enucleated cells express cytoplasmic GFP protein comparable to hT-MSCs (FIG. 1E). Notably, enucleated cells transfected with exogenous Gaussia Luciferase (Gluc) mRNA secreted active Gluc in conditioned medium (CM) at levels similar to parental cells (FIG. 1F). This shows that enucleated cells can translate exogenous mRNAs and secrete functional proteins, demonstrating their functional mRNA translation and protein secretory machineries. Taken together, enucleated cells offer versatile pre- and post-enucleation engineering capabilities that are customizable as a therapeutic platform.

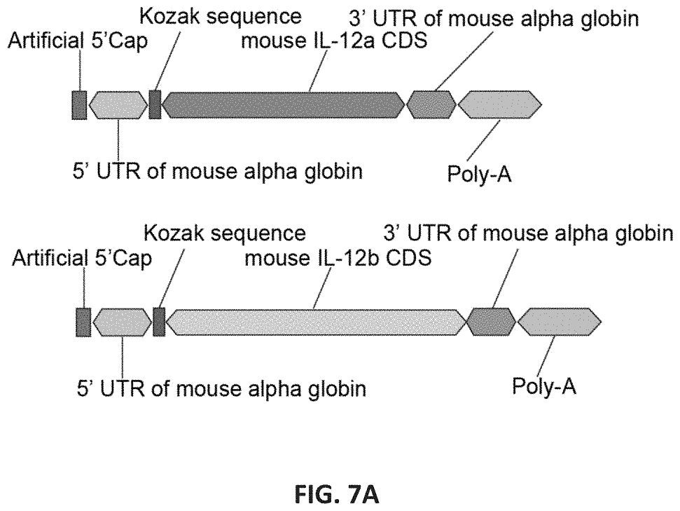

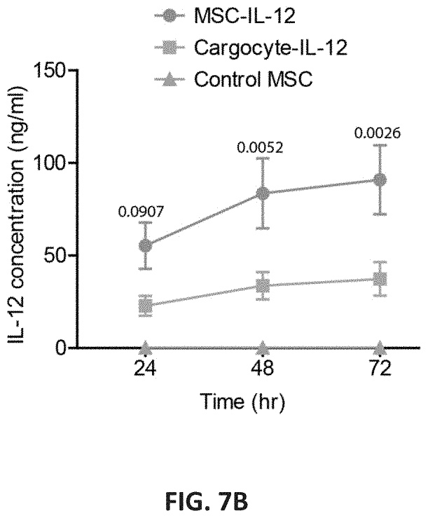

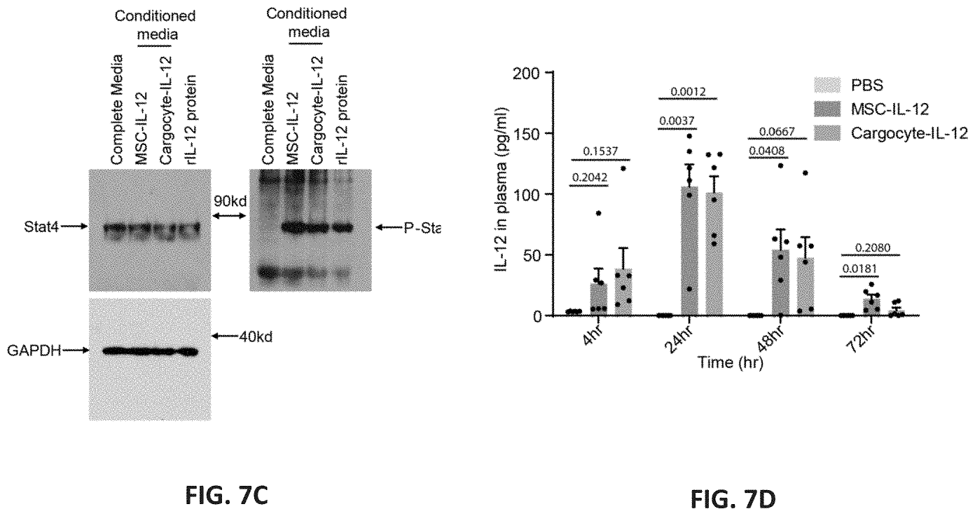

Next, the enucleated cells were engineered to produce therapeutic levels of biologics in vivo. Here, enucleated cells were tested if they can produce bioactive proteins in a tumor microenvironment. The hT-MSCs and enucleated cells were transfected with mouse IL-12 (mIL-12) mRNA (MSC-IL-12 and Cargocyte-IL-12) (FIG. 7A). Enucleated cells-IL-12 secreted mIL-12 in the CM for up to 72 hours as analyzed by ELISA, equating to 20 ng/25,000 enucleated cells per 24 hours (FIG. 7B). Mouse splenocytes treated ex vivo with CM show Stat4 phosphorylation, indicating Cargocyte-IL-12 secrete biologically active mIL-12 (FIG. 7C). To test Cargocyte-IL-12 in vivo therapeutic functions, a mouse model that recapitulates Triple Negative (ER⁻, PR⁻, HER2/neu⁻) Breast Cancer (TNBC), which has a poor prognosis, was used (FIG. 2A). E0771 (mouse TNBC) cells are injected subcutaneously (SQ) in immunocompetent, syngeneic C57BL/6J mice. Tumors developed over 14 days and then Cargocyte-IL-12 or MSC-IL-12 were injected i.t. Cargocyte-IL-12 readily secreted bioactive mIL-12 within the tumor microenvironment (FIG. 2B), induced the transcription of known downstream factors of IL-12 such as IFN-γ, PD-L1, and CXCL9 (FIG. 2C), and recruited key immune effector cells to the tumor site (FIG. 2D). No adverse health issues were noted in treated animals, which was further supported by minimal levels of mIL-12 detected in plasma (FIG. 7D), and no indication of organ dysfunction by hematology. Based on these findings, the ability of Cargocyte-IL-12 to inhibit cancer progression and improve animal survival when used alone or combined with immune checkpoint blockade was determined. Consistent with a recent clinical trial using anti-PD-1 antibodies (aPD-1) combined with in situ vaccination via i.t. (FIG. 15A) IL-12 plasmids delivered by electroporation (NCT03567720), E0771 tumors treated with aPD-1 alone did not affect tumor progression, while tumors treated with Cargocyte-IL-12 followed by aPD-1 had significantly reduced tumor growth and improved animal survival (FIG. 2E), which was comparable to MSC-IL-12 combined with aPD-1. Notably, 40% of animals treated with combined Cargocyte-IL-12 and aPD-1 had complete tumor regression (no palpable tumors) for the remainder of the experiment (>175 days) (FIG. 2E). When these animals were re-challenged with SQ E0771 cells in the contralateral flank, tumors again failed to grow, indicating that the combination therapy produced a durable and systemic antitumor immune response (FIG. 2F). It was also shown that injection of Cargocyte-IL-12 did not negatively impact animal health, and no significant weight changes were noted in animals treated with Cargocyte-IL-12 and PD-1 (FIG. 15B). When Cargocyte-IL-12 was injected IV, changes in acute phase immune markers were minimal to undetectable (FIG. 15C), indicating that Cargocyte-IL-12 are minimally immunogenic. Finally, in a bilateral E0771 TNBC model, tumors were injected unilaterally with 3 doses of Cargocyte-IL-12 or PBS (control), and bilateral tumor size measured over time. Importantly, unilateral injection of Cargocyte-IL-12 reduced tumor growth in both tumors compared to controls (FIG. 15D) and increased tumor infiltration of CD8+ T cells, indicating that Cargocyte-IL-12 injection at a single site can induce a systemic anti-tumor response to limit tumor growth at a distant site (contralateral tumor). Therefore, enucleated cells effectively deliver immunomodulatory biologics to tumor sites and induce systemic anti-tumor immunity with a notable number of animals cured of TNBC.

While nucleated cells like MSCs have been engineered to deliver therapeutic biologics, enucleated cell behavior in vivo is more controllable and predictable because they cannot proliferate or engraft into tissues, and do not have transcriptional machinery that can be activated in the disease microenvironment. In the case of delivering IL-12, IFN-γ is a major downstream effector that can significantly activate gene transcription of undesirable immunosuppressive factors such as PD-L1 and IDO1 on the cell vehicles. When stimulated with human IFN-γ in vitro, both hT-MSCs and irradiated hT-MSCs produced dramatically increased PD-L1 and IDO1 mRNAs, whereas genetically engineered enucleated cells did not (FIG. 2G). Taken together, it is shown that enucleated cells can be bioengineered to effectively deliver immunomodulatory cytokines to diseased tissue through local administration without adverse side effects, which indicates a better safety profile.

### Example 2—In Vivo Homing Ability of Genetically Engineered Enucleated Cells

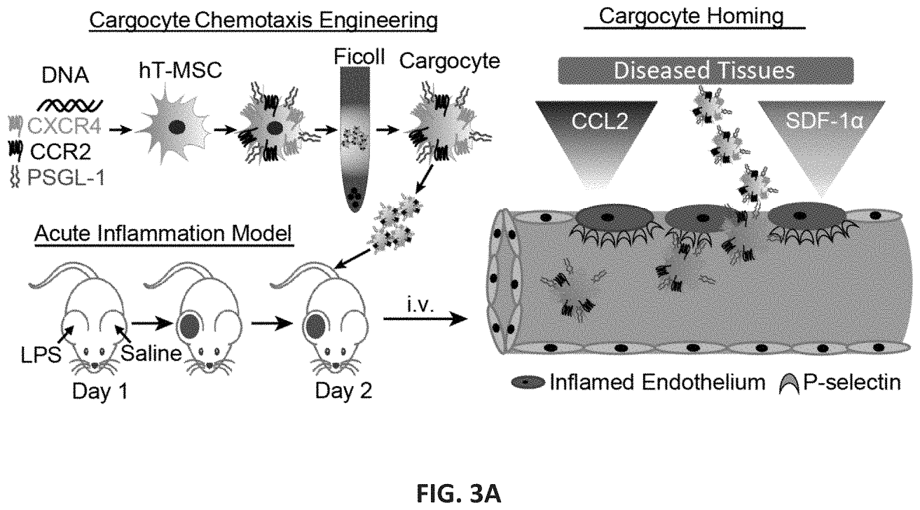

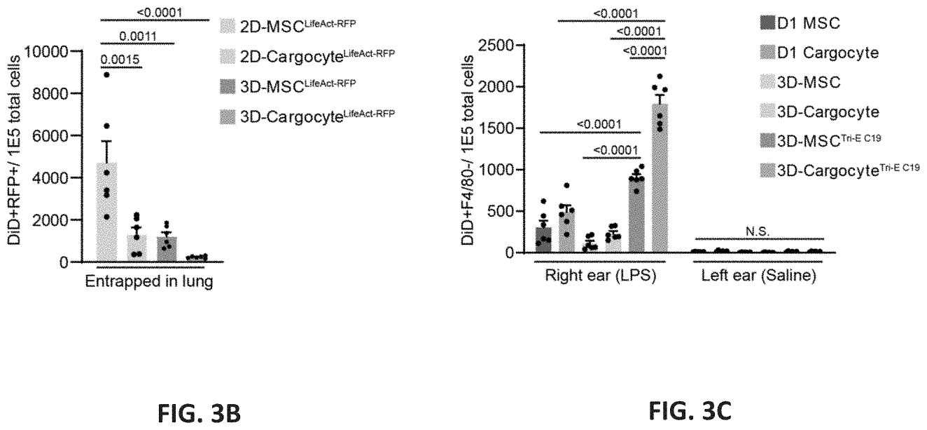

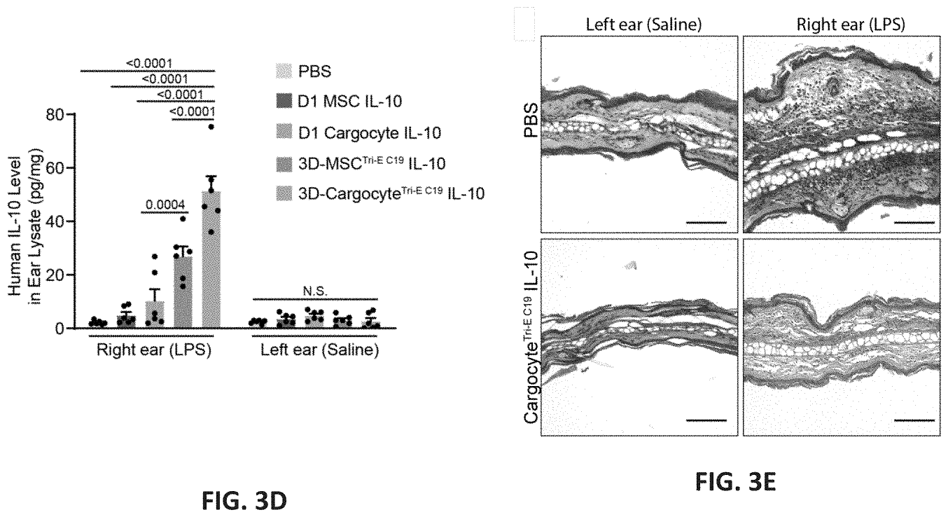

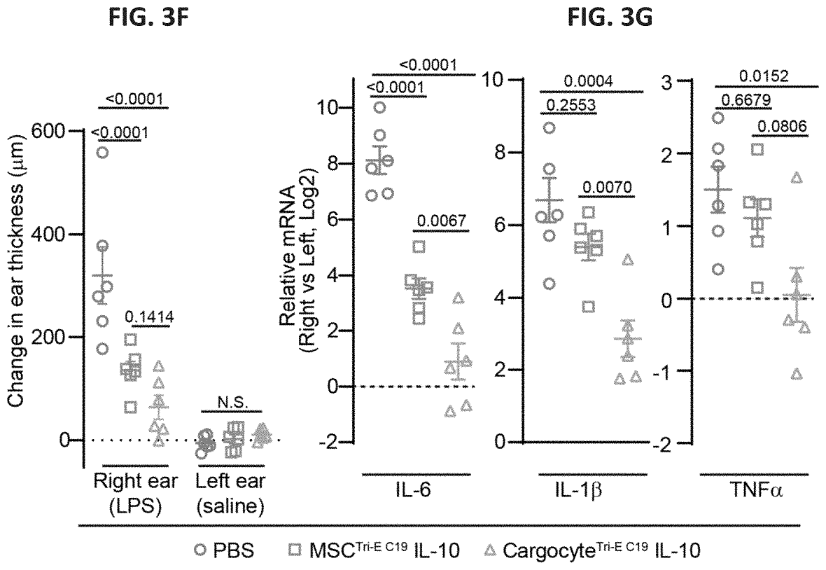

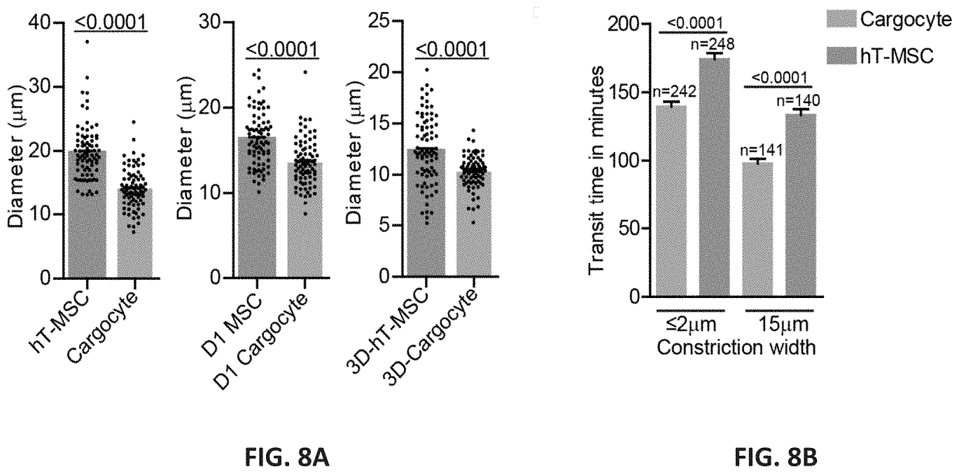

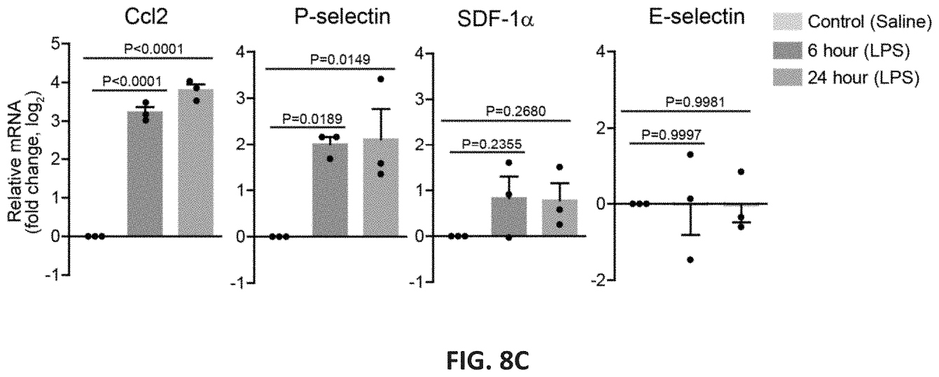

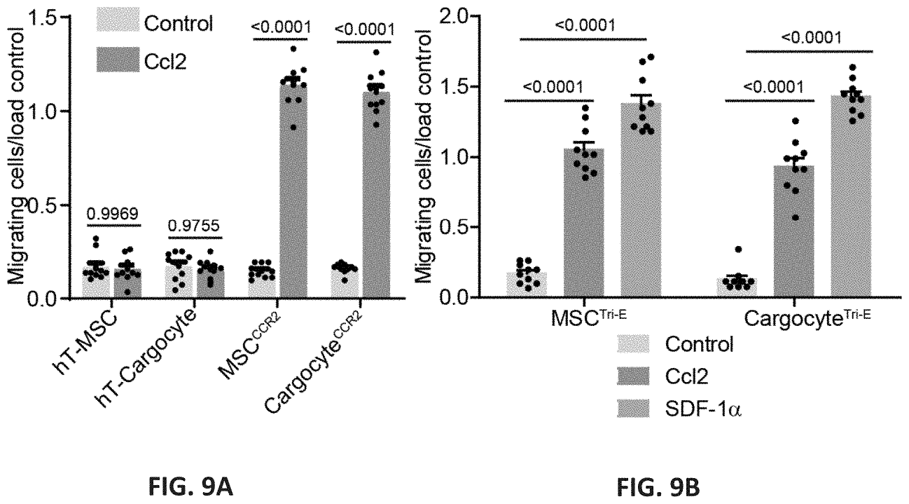

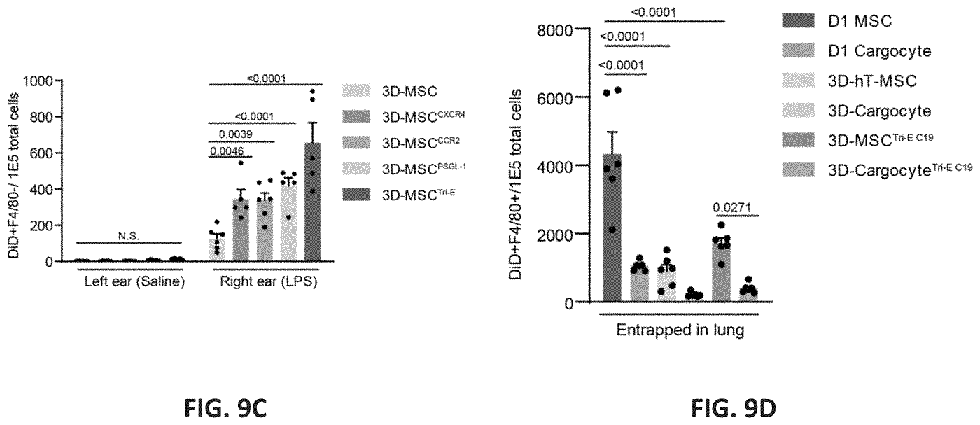

First, since enucleated cells are smaller and lack rigid nuclei (FIG. 8A), enucleated cells were expected to pass through small constrictions such as capillaries or interstitial spaces more effectively than nucleated parental cells. This was tested using a microfluidic device in which LifeAct-RFP-labeled hT-MSCs and enucleated cells were allowed to migrate along a FBS gradient through confined 3D-constrictions that mimic interstitial pores. The migration was imaged by time-lapse confocal microscopy to record the time required for cells to migrate through individual constrictions. As predicted, the enucleated cells efficiently passed through constrictions, whereas hT-MSCs were often trapped in confined constrictions due to the stiff nucleus (FIG. 8B). This result was confirmed in vivo by i.v. injecting mice with hT-MSCs or the enucleated cells double-labeled with LifeAct-RFP and vital dye DiD. By flow cytometry at 24 hours post-injection, there were significantly fewer enucleated cells detected in lung tissue compared to parental cells (FIG. 3B). However, to even further decrease lung entrapment, MSCs were cultured in hanging drops to generate 3D-cultured MSCs that were smaller than traditional 2D-cultured MSCs and showed decreased lung trapping (FIG. 3B) as previously reported. When 3D-cultured MSCs were enucleated, the resulting 3D-Cargocytes were the smallest and had the least lung trapping (FIG. 3B, FIG. 8A and FIG. 9D). Based on these findings, most of the subsequent in vivo homing assays used 3D-hT-MSCs and 3D-Cargocytes. Collectively, our results indicate enucleated cells pass through microvasculature better than parental cells, which may facilitate better in vivo homing.

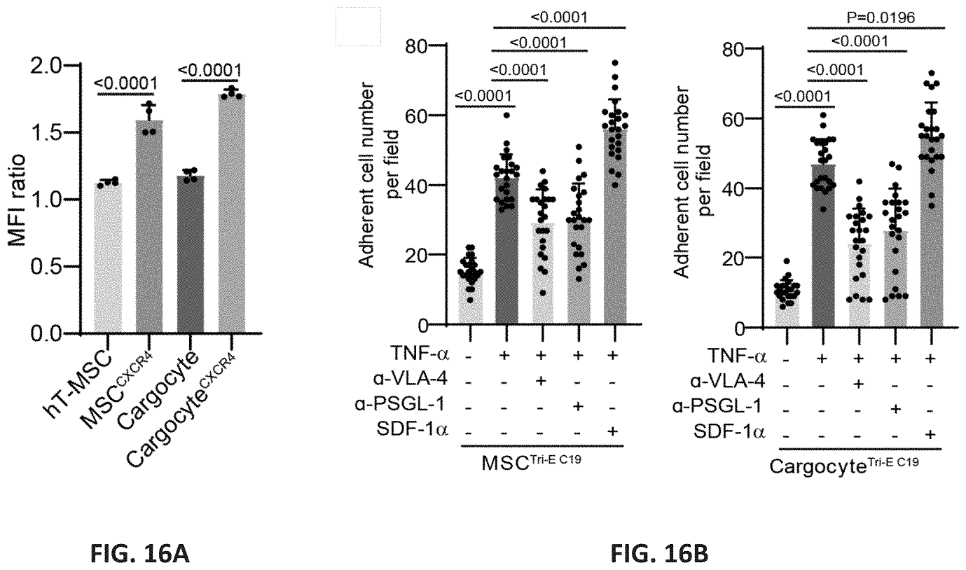

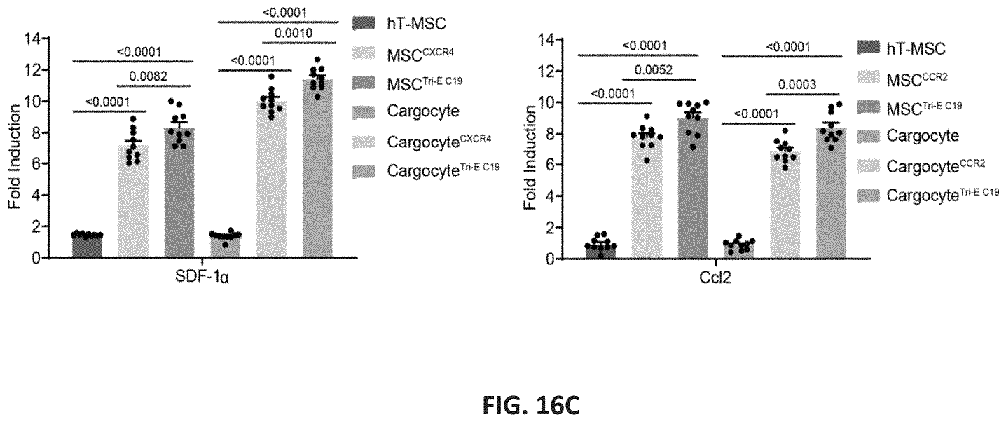

Next, the enucleated cells were designed and engineered with specific chemokine receptors and adhesion molecules corresponding to a diseased tissue, which was hypothesized to increase enucleated cells homing to the target site in vivo. An acute inflammation mouse model was used, in which bacterial-derived lipopolysaccharide (LPS) was intradermally (i.d.) injected into the pinna to induce acute, local inflammation. Saline was i.d. injected into the contralateral ear as a control. This model allows examination of therapeutic cell homing quantitatively between an inflamed and non-inflamed contralateral tissue within the same animal. It was found that SDF-1α, Ccl2, and P-Selectin but not E-Selectin were upregulated in inflamed ears compared to controls, starting 6 hours post-LPS injection (FIG. 8C). Then the hT-MSCs were engineered to stably express either CXCR4 (MSC^{CXCR4}) to bind SDF-1α, CCR2 (MSC^{CCR2}) to bind Ccl2, or the endothelial adhesion molecule PSGL-1 with fucosyltransferase 7 (FUT-7, for functional modification of PSGL-1) (MSC^{PSGL-1}) to bind P- and E-selectins. Each of these engineered MSCs was enucleated to create the corresponding enucleated cells (Cargocyte^{CXCR4}, Cargocyte^{CCR2}, and Cargocyte^{PSGL-1}). Flow cytometry showed engineered enucleated cells retained stable surface expression of CXCR4, CCR2 or PSGL-1 for at least 48 hours post-enucleation. Functionally, Cargocyte^{PSGL-1} showed increased binding to its receptors P-/E-selectin up to 48 hours post-enucleation. Just as Cargocyte^{CXCR4} responded robustly to SDF-1α (FIG. 1D), Cargocyte^{CCR2} showed dramatic chemotaxis towards Ccl2 compared to non-engineered enucleated cells (FIG. 9A). Finally, using 3 separate DNA-integrating lentiviruses under different drug selection, hT-MSCs were engineered to simultaneously express CCR2, CXCR4, and PSGL-1/FUT-7, then FACS sorted to enrich for cells with high expression of all 3 markers (named MSC^{Tri-E}). MSC^{Tri-E}-derived Cargocyte^{Tri-E} showed robust migration towards Ccl2 and SDF-1α gradients (FIG. 9B, FIG. 16A-16C), indicating that co-expression of engineered receptors improved migration without functionally interfering with each other.

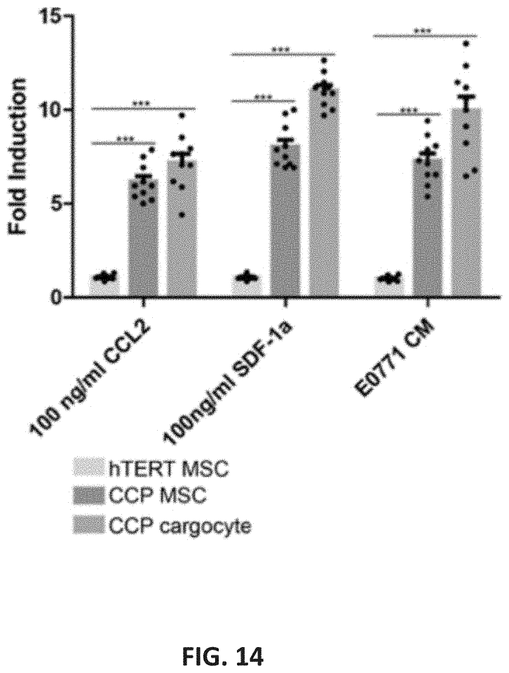

Also, enucleated cells were engineered with leukocyte homing molecules combined with their innate tumor trophic properties, which was hypothesized to significantly improve enucleated cells homing to tumors. It was determined if CCP cargocytes can home to chemokines produced by E0771 murine BC conditioned media (CM). E0771 cells are an established murine BC line that produce SDF-1aα and CCL2 in vitro and in tumors in vivo. The engineered enucleated cells migrates towards E0771 CM, which is significantly enhanced through genetic engineering with CXCR4 and CCR2 chemoattractant receptors (FIG. 14).

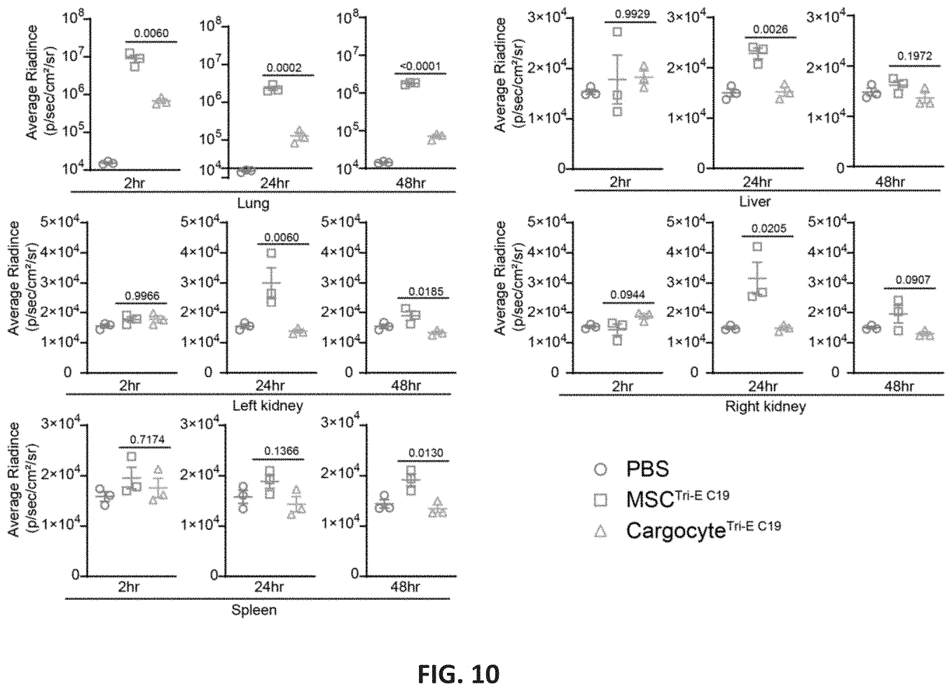

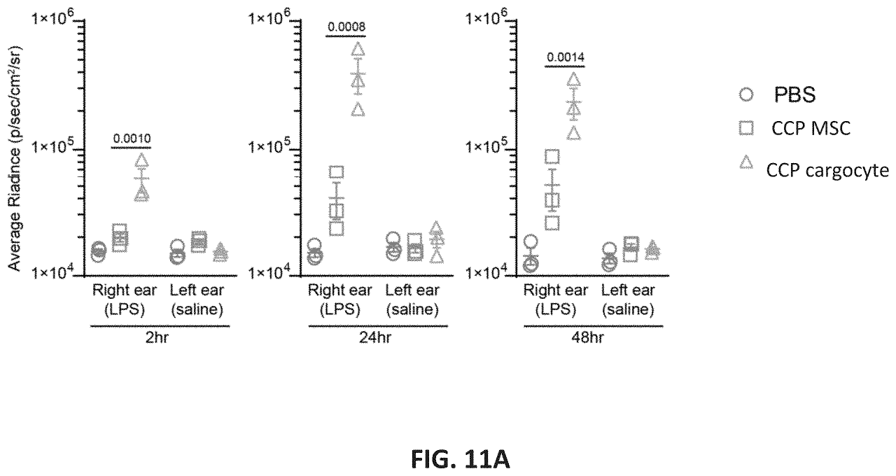

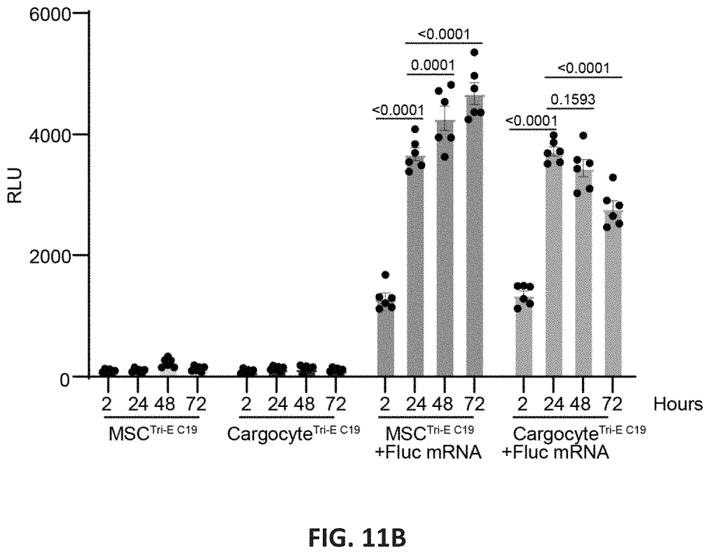

It was then tested if the engineering strategy improved in vivo homing. 3D-cultured MSCs were labeled with DiD and i.v. injected into mice 6 hours after i.d. LPS injection (FIG. 3A). Mouse tissues were harvested 24 hours after i.v. injection and analyzed by flow cytometry for DiD⁺F4/80⁻ cells, which excludes the possibility of non-specific DiD incorporation into mouse macrophages. Individual expression of CCR2, CXCR4, or PSGL-1 improved cell homing specifically to the inflamed ear compared to non-engineered hT-MSCs, indicating that these proteins are functional in vivo and contribute to homing. Notably, MSC′ simultaneously expressing all 3 surface proteins showed the greatest homing (FIG. 9C), suggesting multiple layers of engineering can be combined to achieve superior in vivo homing. To further increase the consistency and homogeneity of engineered cells, FACS was used to sort MSC^{Tri-E} to establish 19 single cell MSC^{Tri-E} clones with high expression of all 3 markers, and selected Clone 19 (MSC^{Tri-E C19}) for subsequent in vivo experiments based on surface expression, growth rate, and cell size. 3D-MSC^{Tri-E C19} were enucleated to generate 3D-Cargocyte^{Tri-E C19}, which showed superior homing to inflamed ears compared to non-engineered 3D-Cargocytes (FIG. 3C). Since both engineered and non-engineered 3D-Cargocytes had similar low lung trapping (FIG. 9D) but only engineered enucleated cells had significantly improved homing to the ear, our results suggest that engineered surface proteins on 3D-Cargocyte^{Tri-E C19} significantly improved in vivo homing. Additionally, 3D-Cargocyte^{Tri-E C19} also showed significantly better homing compared to mouse D1 MSCs or enucleated cells (FIG. 3C), which are a syngeneic MSC line from BALB/c mice that possess endogenous homing abilities. This result was independently confirmed by a bioluminescence assay using firefly luciferase that showed 3D-Cargocyte^{Tri-E C19} had decreased lung retention but dramatically increased homing to the inflamed ear compared to 3D-MSC^{Tri-E C19} as early as 2 hours post i.v. injection, indicating the specific homing of engineered Cargocytes to the designated disease tissue with minimal accumulation in other organs (FIG. 10, FIGS. 11A and 11B). Importantly, by immunostaining with specific antibodies against human mitochondria and nuclei, i.v. injected 3D-Cargocyte^{Tri-E C19} were detected outside of vessel lumina and within the ear connective tissue, indicating that enucleated cells were not passively trapped in the ear vasculature, but were able to extravasate into tissues. Together, these data show for the first time that enucleated cells can be extensively engineered to specifically home to designated diseased tissue following systemic administration. The enucleated cells also exhibit better homing efficiency than parental cells because of decreased entrapment in the lungs.

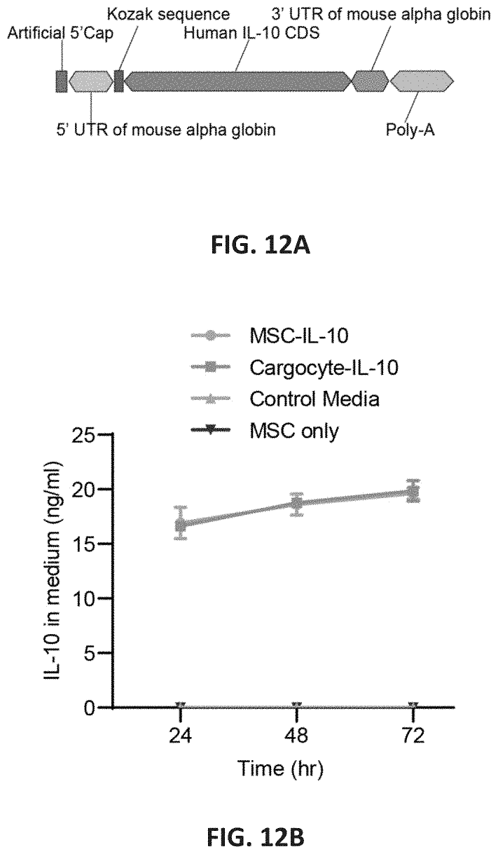

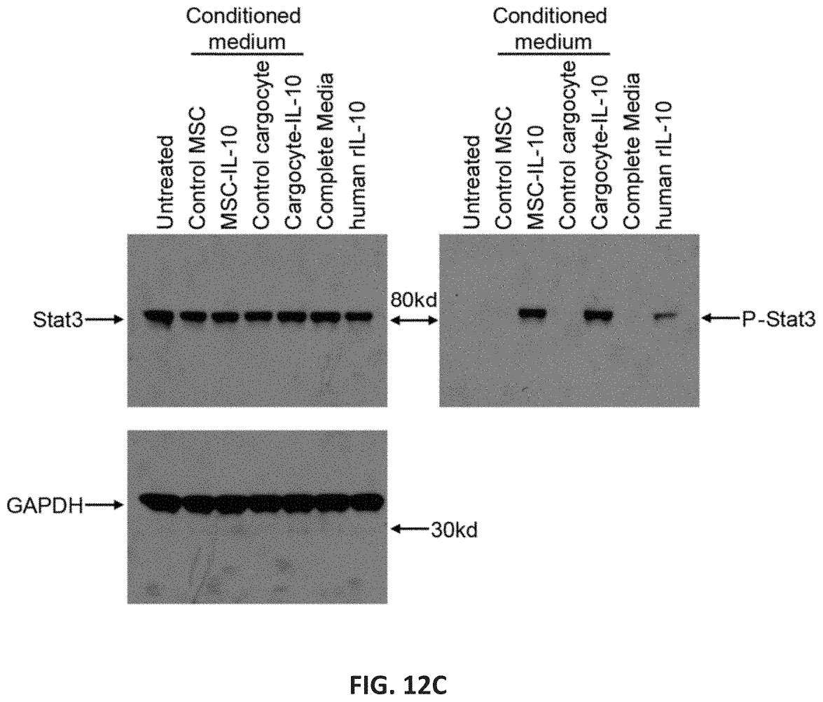

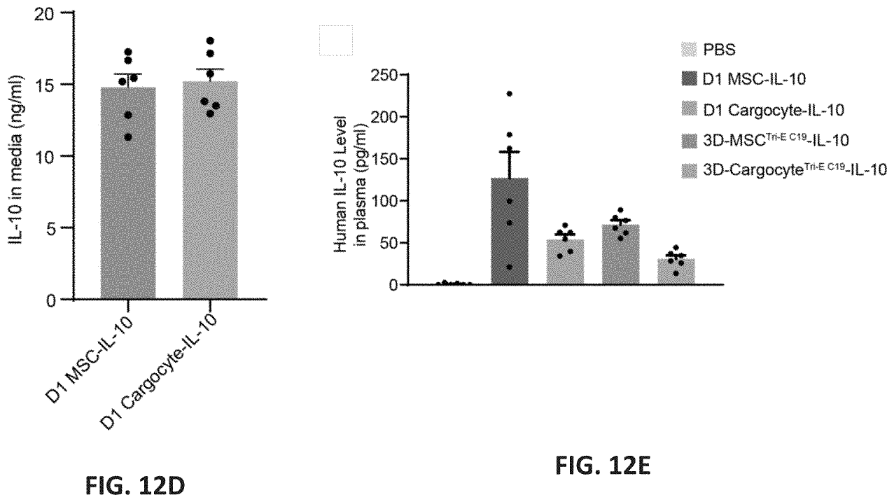

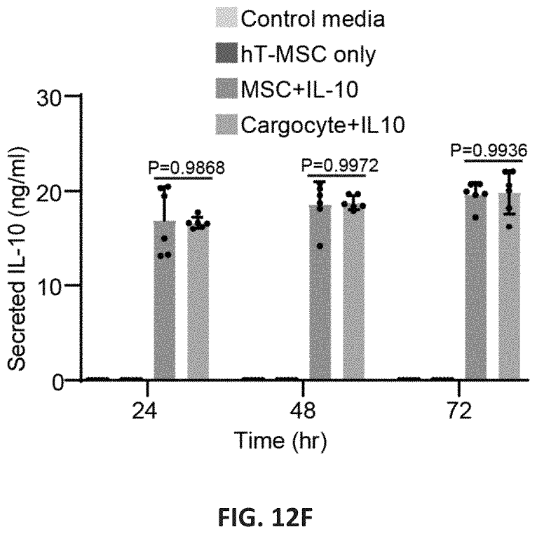

Next, the ability of bioengineered enucleated cells (e.g., cargocytes) to deliver anti-inflammatory biologics to treat inflamed tissue was investigated. IL-10 is a potent anti-inflammatory cytokine, but clinical applications need more efficient and specific delivery methods. Enucleated cells transfected with human IL-10 mRNA (Cargocyte-IL-10) produced IL-10 for up to 72 hours in vitro, similar to transfected parental cells (MSC-IL-10), while non-engineered hT-MSCs did not secrete detectable IL-10 (FIGS. 12A and 12B). Functionally, CM from MSC-IL-10 and Cargocyte-IL-10 activated Stat3 phosphorylation in mouse RAW macrophages in vitro, indicating the secreted hIL-10 was biologically active on mouse cells (FIG. 12C). While the level of hIL-10 secretion in vitro was comparable between all the types of cells and enucleated cells (FIGS. 12D and 12F), injection of 3D-Cargocytes^{Tri-E C19} resulted in the highest levels of hIL-10 in the ear (FIG. 3D), likely because their superior homing to the ear allowed for effective delivery at the intended site. All contralateral (control) ears from these animals had barely detectable hIL-10 (FIG. 3D), suggesting the delivery of hIL-10 to inflamed ears was specific.

Then the therapeutic efficacy of engineered enucleated cells in the inflamed tissue was examined. Histologically, inflamed ears from PBS-treated mice showed severe hemorrhage and edema with moderate amounts of mixed leukocytes, while mice treated with 3D-Cargocytes^{Tri-E} IL-10 had minimal hemorrhage and edema and mild amounts of mixed leukocytes (FIG. 3E). In this ear inflammation model, the increased fluid and cellular infiltration correspond to an increased thickness of the pinna, which can be measured as a marker of degree of inflammation. While the thickness of saline-injected control ears was static and comparable across all groups, both engineered MSC and enucleated cell-treated animals had significantly thinner inflamed ears compared to control mice (FIG. 3F). Additionally, the expression of inflammation markers IL-6, IL-1β and TNF-α were significantly downregulated in ears of mice treated with 3D-Cargocytes^{Tri-E C19} IL-10 compared to PBS-treated mice (FIG. 3G). Therefore, bioengineered enucleated cells that are small in size and express homing receptors and endothelial adhesion molecules can specifically home to designated inflamed tissues and effectively deliver anti-inflammatory cytokines, overall reducing inflammation in vivo.

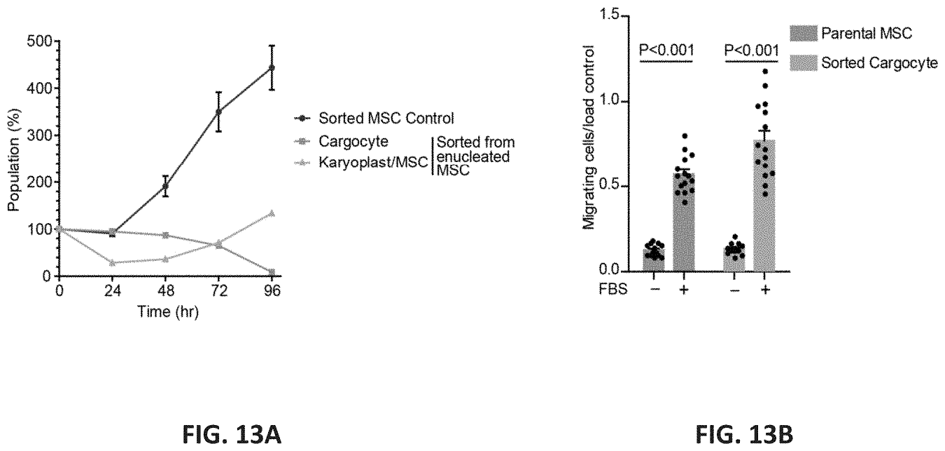

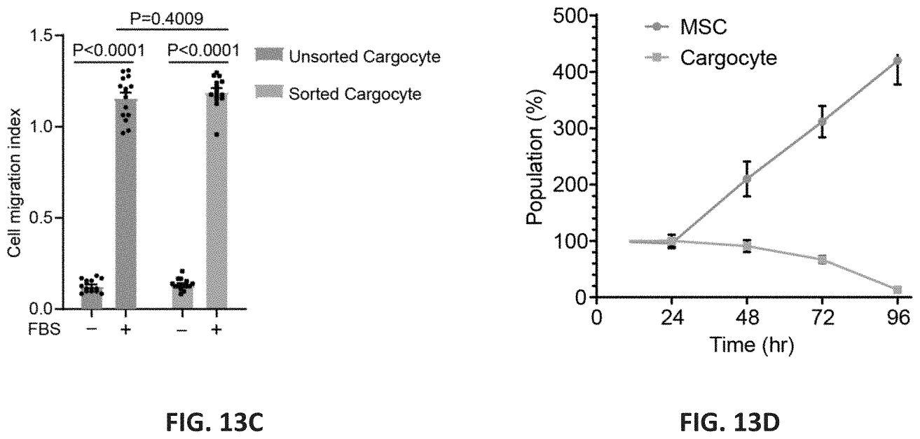

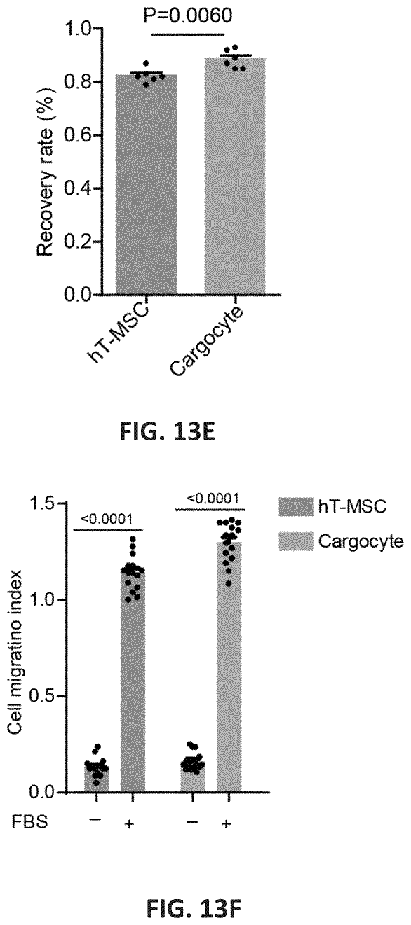

Finally, it was shown that engineered human MSC-derived enucleated cells in mouse models have not produced any overt negative health effects in over 300 mice following i.t. or i.v. administration, as determined by clinical observation and gross examination of tissues by a board-certified veterinary pathologist (C.N.A.). BALB/c mice i.v. injected with bioengineered enucleated cells (e.g., cargocytes) had no significant change in the plasma concentration of pro-inflammatory cytokines IL-6, IL-1β, TNF-α and IFN-γ at 4 hours or 24 hours post-injection. Moreover, as a prototype for clinical use, we labeled cell nuclei with Histone 2B-GFP and generated Cargocytes of 99.999% purity through FACS, without loss of viability or migration ability (FIG. 13A-13F). Based on these results, enucleated cells have the potential to be a safe and effective therapeutic platform.

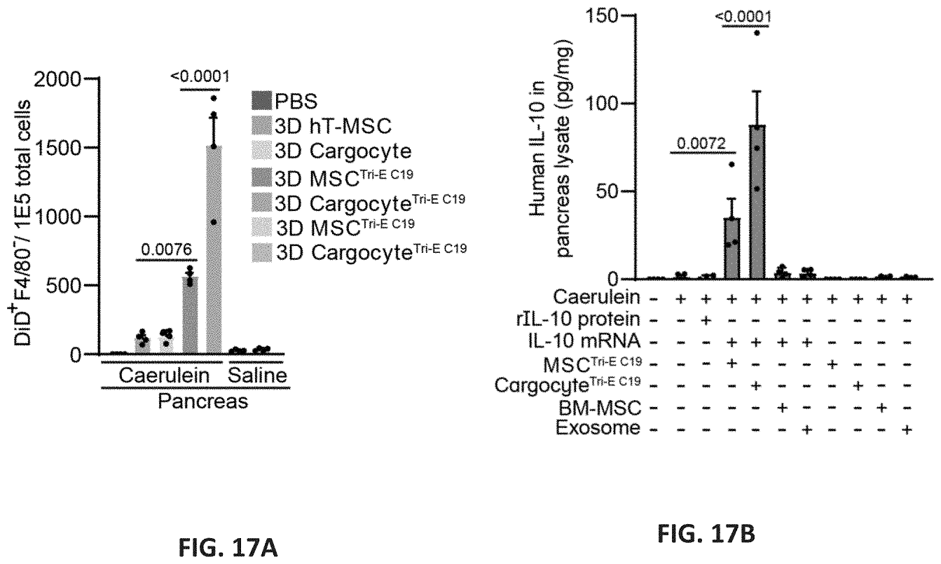

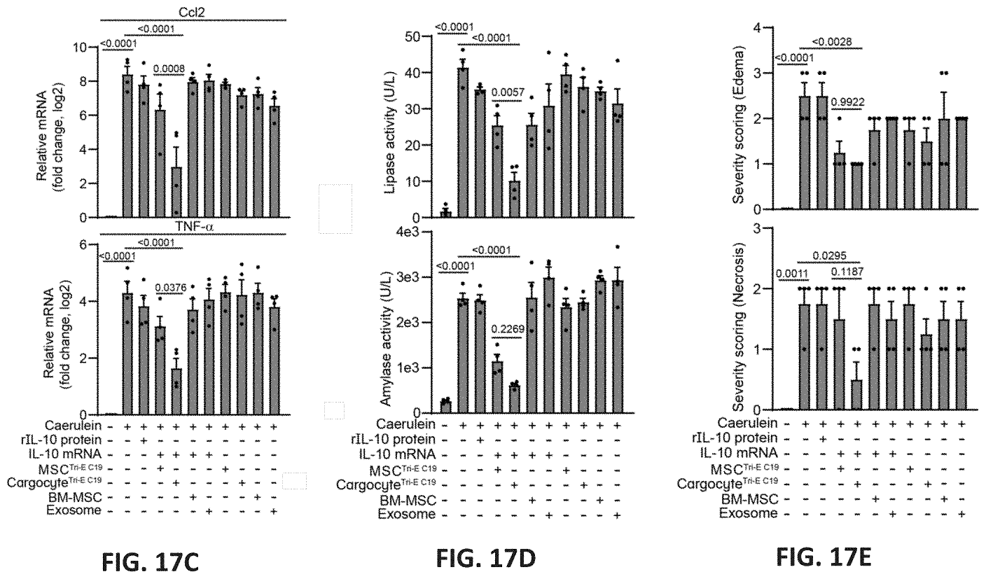

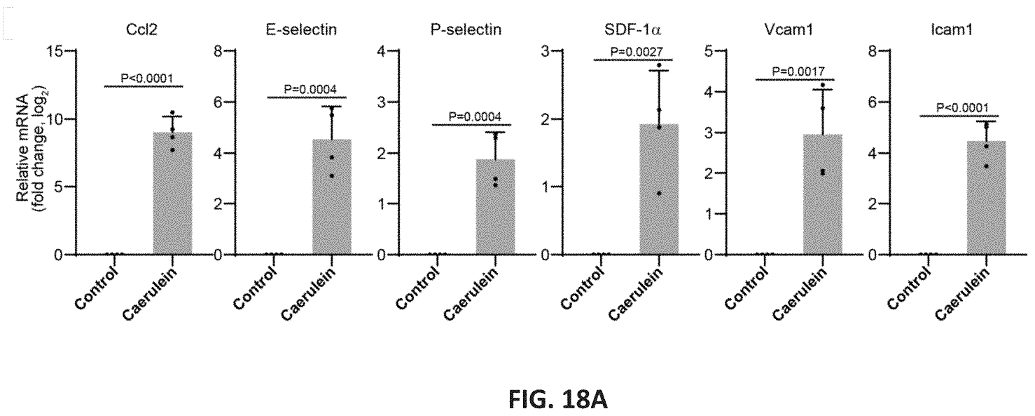

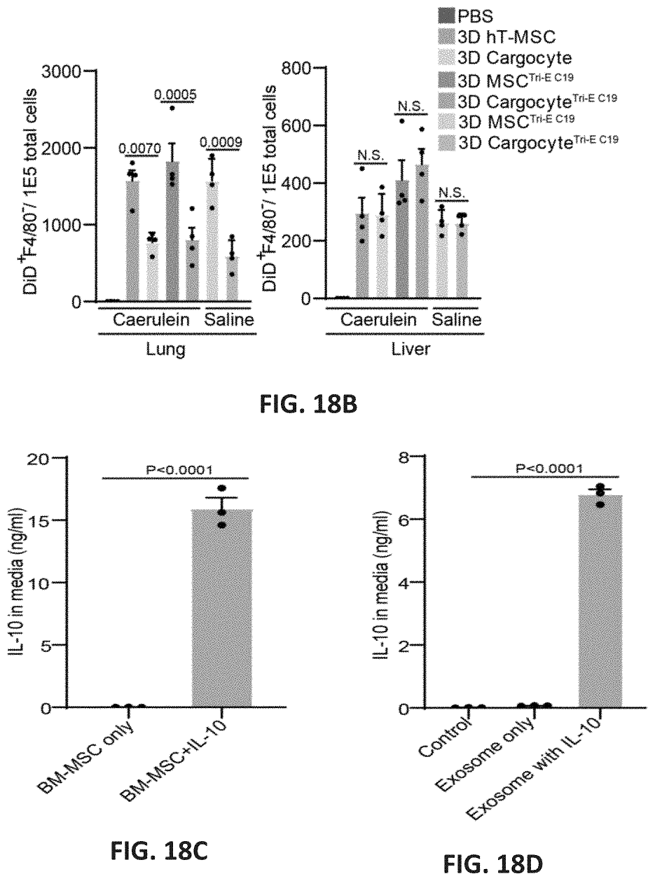

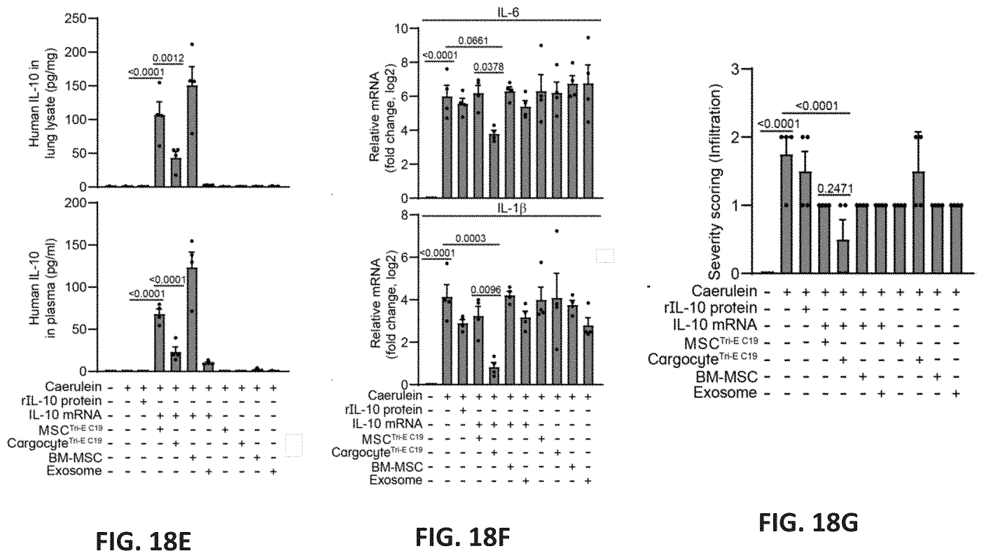

Further, the therapeutic delivery of bioengineered enucleated cells in a disease model of acute pancreatitis (AP) was tested. AP is a severe disease with significant morbidity and mortality that currently lacks effective treatments. Caerulein is a decapeptide analog of hormone Cholecystokinin (CCK), which can stimulate exocrine pancreatic secretion and induce AP in pre-clinical mouse models. Previous studies suggested frequent systemic administration of high doses of anti-inflammatory cytokine IL-10 in pre-clinical AP models can greatly attenuate the inflammation and mitigate the disease. However, repeated high doses of IL-10 are not cost-effective in clinical applications and may also lead to unwanted severe complications such as anemia, suggesting a specific and efficient delivery vehicle may be necessary. In early stage of caerulein-induced AP, chemokines such as Ccl2 and SDF-1α, and adhesion molecules such as E-/P-Selectins and Vcam1, are all significantly upregulated in the inflamed mouse pancreas (FIG. 18A), suggesting bioengineered CargocytesTri-E C19 may be an ideal delivery vehicle to specifically deliver IL-10 to the inflamed pancreas. The homing of Cargocytes and parental MSCs in AP mice was assessed by Vybrant-DiD labeling and FACS analysis. Compared to non-engineered 3D-Cargocytes, 3D-CargocytesTri-EC19 homed more efficiently (>11-fold) to the inflamed pancreas (FIG. 17A). 3D-CargocyteTri-E C19 also had a >2-fold increase in homing to the inflamed pancreas and decreased lung trapping compared to parental 3D-MSCTri-E C19 (FIG. 17A and FIG. 18B). In the healthy non-treated pancreas, both 3D-CargocyteTri-E C19 and 3D-MSCTri-E C19 had minimum accumulation. Importantly, compared to parental 3D-MSCTri-E C19, 3D-CargocyteTri-E C19 also delivered IL-10 protein more efficiently (>2-fold) to the inflamed pancreas (FIG. 17B), which correlated with decreased expression of the inflammatory gene markers, Ccl2, TNF-α, IL-1β, and IL-6 (FIG. 17C and FIG. 18F). Infusion of 3D-CargocyteTri-E C19 IL-10 also significantly reduced blood serum levels of lipase and amylase (FIG. 17D), which correlate with the severity of pancreas damage. And histological analyses showed reduced acinar cell necrosis, lower interstitial edema, and less inflammatory cell infiltration in the damaged pancreas (FIG. 17E and FIG. 18G). Notably, 3D-CargocyteTri-E C19 or 3D-MSCTri-E C19 without IL-10 did not significantly affect caerulein-induced pancreatitis (FIG. 17B-17E). Together, these results demonstrate that bioengineered CargocyteTri-E C19 can efficiently deliver bioactive anti-inflammatory cytokine IL-10 to the inflamed pancreas, which greatly ameliorates the disease in an established clinically relevant AP model. As expected, animals i.v. injected with a single dose (8 μg/kg body weight) of recombinant hIL-10 protein had minimal effects in all inflammatory and AP markers, probably due to the short half-life of IL-10 protein in circulation (FIG. 17B-17E). Also, as a comparison to recognized therapeutic delivery platforms, commercially available bone marrow-derived primary MSCs (BM-MSC) and purified BM-MSC-derived exosomes (B-exosomes) loaded with IL-10 mRNAs in the AP model. While infusion of BM-MSCs+IL-10 significantly increased the level of IL-10 in the serum and in the lung (FIG. 18E), the actual IL-10 levels detected in the inflamed pancreas were negligible (FIG. 17B), suggesting that BM-MSCs are inefficient at homing and delivering IL-10 to the inflamed pancreas. Similarly, animals injected with B-exosomes loaded with IL-10 mRNA showed no detectable IL-10 in the inflamed pancreas and only a slight increase in serum IL-10 levels (FIG. 17B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a cancer in a subject, comprising: administering to the subject:
a therapeutically effective amount of a formulation comprising a plurality of enucleated cells, wherein at least one enucleated cell of the plurality of enucleated cells comprises a polypeptide comprising a cytokine expressed by the at least one enucleated cell from an mRNA exogenous to the at least one enucleated cell; and
a therapeutically effective amount of an immune checkpoint inhibitor,
wherein the polypeptide and the immune checkpoint inhibitor are a combination therapy for treatment of the cancer.

2. The method of claim 1, the cytokine comprises an interleukin.

3. The method of claim 2, wherein the interleukin comprises interleukin 12 (IL-12).

4. The method of claim 1, wherein the immune checkpoint inhibitor comprises a PD-1 inhibitor, a PD-L1 inhibitor, a TIM-3 inhibitor, a LAG-3 inhibitor, a TIGIT inhibitor, a CD47 inhibitor, a B7 inhibitor, a CD 137 inhibitor, a CTLA-4 inhibitor, or any combination thereof.

5. The method of claim 4, wherein the immune checkpoint inhibitor comprises the PD-L1 inhibitor.

6. The method of claim 1, wherein the at least one enucleated cell further comprises the immune checkpoint inhibitor.

7. The method of claim 6, wherein the at least one enucleated cell further comprises an additional exogenous mRNA encoding the immune checkpoint inhibitor.

8. The method of claim 1, wherein the immune checkpoint inhibitor is encoded by at least one additional exogenous RNA in at least one additional enucleated cell.

9. The method of claim 1, wherein the at least one enucleated cell is depleted of an immune recognition molecule.

10. The method of claim 9, wherein the immune recognition molecule comprises HLA antigen, proteoglycan, sugar moiety, embryonic antigen, or any combination thereof.

11. The method of claim 1, further comprising administering one or more additional therapeutic agents, wherein the one or more additional therapeutic agents comprise chemotherapy, cell-based therapy, radiation therapy, immunotherapy, a small molecule therapy, an inhibitory nucleic acid therapy, or any combination thereof.

12. The method of claim 1, wherein the administering comprises intravenous administration, subcutaneous administration, intraperitoneal administration, rectal administration, oral administration, intratumoral administration, intramuscular administration, inhalation administration, retroperitoneal administration, or combinations thereof.

13. The method of claim 12, wherein the administering comprises intratumoral administration.

14. The method of claim 1, wherein the cancer comprises multiple myeloma, glioblastoma, lymphoma, leukemia, mesothelioma, sarcoma, breast cancer, prostate cancer, ovarian cancer, pancreatic cancer, colon cancer, or combinations thereof.

15. A therapeutic formulation, comprising: a plurality of enucleated cells, wherein at least one enucleated cell of the plurality of enucleated cells comprises a polypeptide comprising a cytokine, wherein the polypeptide is formulated to be administered to a subject as a combination therapy comprising a therapeutically effective amount of an immune checkpoint inhibitor, and wherein the combination therapy is therapeutically effective to a treat cancer in the subject.

16. The therapeutic formulation of claim 15, wherein the cytokine comprises an interleukin.

17. The therapeutic formulation of claim 16, wherein the interleukin comprises IL-12.

18. The therapeutic formulation of claim 15, wherein the immune checkpoint inhibitor comprises a PD-1 inhibitor, a PD-L1 inhibitor, a TIM-3 inhibitor, a LAG-3 inhibitor, a TIGIT inhibitor, a CD47 inhibitor, a B7 inhibitor, a CD 137 inhibitor, a CTLA-4 inhibitor, or any combination thereof.

19. The therapeutic formulation of claim 18, wherein the immune checkpoint inhibitor comprises the PD-L1 inhibitor.

20. The therapeutic formulation of claim 15, wherein the at least one enucleated cell further comprises the immune checkpoint inhibitor.

21. The therapeutic formulation of claim 20, wherein the at least one enucleated cell further comprises an additional exogenous mRNA encoding the immune checkpoint inhibitor.

22. The therapeutic formulation of claim 15, wherein the immune checkpoint inhibitor is encoded by at least one additional exogenous RNA in at least one additional enucleated cell.

23. The therapeutic formulation of claim 15, wherein the enucleated cell is depleted of an immune recognition molecule.

24. The therapeutic formulation of claim 23, wherein the immune recognition molecule comprises a HLA antigen, proteoglycan, sugar moiety, embryonic antigen, or a combination thereof.

25. The therapeutic formulation of claim 15, wherein the therapeutic formulation is formulated for single dose administration.

26. The therapeutic formulation of claim 15, wherein the therapeutic formulation is formulated for sequential administration.

27. The therapeutic formulation of claim 15, wherein the therapeutic formulation is formulated for intravenous administration, subcutaneous administration, intraperitoneal administration, rectal administration, oral administration, intratumoral administration, intramuscular administration, inhalation administration, retroperitoneal administration, or combinations thereof.

28. The therapeutic formulation of claim 27, wherein the therapeutic formulation is formulated comprises intratumoral administration.

29. The therapeutic formulation of claim 15, wherein the cancer comprises multiple myeloma, glioblastoma, lymphoma, leukemia, mesothelioma, sarcoma, breast cancer, prostate cancer, ovarian cancer, pancreatic cancer, colon cancer, or combinations thereof.
